(19) United States
(12) Patent Application Publication
(10) Pub. No.: US 2025/0388842 A1
(43) Pub. Date: Dec. 25, 2025

(54) MULTI-ORGAN MICROFLUIDIC CHIP

(71) Applicant: SynVivo Inc., Huntsville, AL (US)

(72) Inventors: Gwendolyn Diedre Fewell, Madison, AL (US); Deborah Michele Ramsey, Huntsville, AL (US); Jenna Marie Rosano, Huntsville, AL (US)

(21) Appl. No.: 19/304,374

(22) Filed: Aug. 19, 2025

Related U.S. Application Data
(63) Continuation-in-part of application No. PCT/US2025/032720, filed on Jun. 6, 2025.
(60) Provisional application No. 63/657,562, filed on Jun. 7, 2024.

(51) Int. Cl. C12M 3/06; C12M 1/00; C12M 1/34; G01N 33/50

(52) U.S. Cl. CPC C12M 23/16; C12M 23/20; C12M 23/34; C12M 23/58; C12M 29/14; C12M 29/20; C12M 41/34; C12M 41/36; G01N 33/5023; G01N 33/5026; G01N 33/5029; G01N 33/5047; G01N 33/5088

(57) ABSTRACT

A microfluidic chip may comprise a plurality of separate microfluidic regions formed into a chip substrate. Each microfluidic region may be configured to simulate an organ and may comprise at least one inlet and at least one outlet with a microfluidic channel therebetween. Connecting microfluidic pathways may be provided between the separate microfluidic regions. The connecting microfluidic pathways may be configured to be vascularized. The connecting microfluidic pathways may comprise a surface treatment configured to receive endothelial cells. The surface treatment may comprise an extracellular matrix protein coating. Endothelial cells may be disposed within the connecting microfluidic pathways. The endothelial cells may comprise organ-specific endothelial cells that match an organ simulated by at least one of the separate microfluidic regions. The connecting microfluidic pathways may comprise synthetic microvascular networks having non-linear channels with physiologically relevant geometries. The physiologically relevant geometries may comprise bifurcations, varying cross-sectional areas, and convolutions.

### CROSS-REFERENCE TO RELATED APPLICATIONS

This patent application is a continuation-in-part of PCT Application No. PCT/US2025/032720 filed Jun. 6, 2025, which claims priority to U.S. Provisional Application No. 63/657,562 filed Jun. 7, 2024, which applications are incorporated herein by specific reference in their entirety.

### BACKGROUND

### Field

The present disclosure relates to devices having multiple organ-simulating regions with vascularized connections for biological assays.

### Description of Related Art

The study and simulation of microvascular networks have been pivotal in advancing biomedical research, particularly in understanding cellular behaviors, drug delivery mechanisms, and disease progressions. Microfluidic devices have emerged as essential tools in replicating the complex environments of human vasculature, enabling precise control over fluid dynamics and cellular interactions.

Several innovations have been introduced in this domain. For instance, US20070231783A1 discloses a microfluidic device designed to mimic microvascular networks, facilitating the study of blood flow and cellular responses under controlled conditions. Similarly, US20100112550A1 presents a microfluidic assay that characterizes leukocyte adhesion, providing insights into inflammatory responses within vascular structures.

Advancements have also been made in creating idealized microvascular networks, as seen in US20130149735A1, which offers a platform for studying particle adhesion and cellular dynamics in bifurcated microchannels. US20100227312A1 further explores particle adhesion assays within microfluidic bifurcations, enhancing our understanding of particle behavior in microcirculatory systems.

The integration of microfluidic networks into cell culture devices has been addressed in US20150377861A1, which describes a cell culture assay device comprising a substrate with multiple discrete microfluidic networks and wells, allowing for high-throughput analysis of cellular responses. US20150299631A1 introduces a multi-chambered cell culture device that models organ systems by simulating various tissue interfaces, providing a more comprehensive in vitro environment.

Furthermore, US20140255961A1 discusses synthetic microfluidic systems tailored for wound healing and hemostasis studies, emphasizing the therapeutic applications of microfluidic technologies in regenerative medicine.

Despite these significant contributions, there remains a need for more versatile and integrative microfluidic platforms that can accurately replicate the multifaceted interactions within human tissues and organs. Current systems often face limitations in scalability, modularity, and the ability to simulate complex physiological conditions. Therefore, there is a continued demand for innovative microfluidic devices that offer enhanced simulation of human biological systems, facilitating more accurate and comprehensive biomedical research.

### SUMMARY

In some embodiments, a microfluidic chip may comprise a plurality of separate microfluidic regions formed into a chip substrate, each microfluidic region configured to simulate an organ and comprising at least one inlet and at least one outlet with a microfluidic channel therebetween. The microfluidic chip may further include connecting microfluidic pathways between the separate microfluidic regions, wherein the connecting microfluidic pathways may be configured to be vascularized. The connecting microfluidic pathways may comprise a surface treatment configured to receive endothelial cells. The surface treatment may comprise an extracellular matrix protein coating. Endothelial cells may be disposed within the connecting microfluidic pathways. The endothelial cells may comprise organ-specific endothelial cells that match an organ simulated by at least one of the separate microfluidic regions. The microfluidic chip may include a first cell type of a first organ in a first microfluidic region configured for simulating the first organ, and a second cell type of a second organ in a second microfluidic region configured for simulating the second organ, wherein the first organ may be different from the second organ. The connecting microfluidic pathways may comprise synthetic microvascular networks (SMNs) having non-linear channels with physiologically relevant geometries. The physiologically relevant geometries may comprise bifurcations, varying cross-sectional areas, and convolutions. Alternatively, the connecting microfluidic pathways may comprise idealized microvascular networks (IMNs) having linear channels with uniform dimensions.

At least one of the separate microfluidic regions may comprise a porous wall between at least two microfluidic channels to allow molecular exchange while maintaining separation between different cell types. The microfluidic chip may further comprise at least one electrode set, an oxygen sensor, or a camera operably coupled with at least one of the separate microfluidic regions.

A microfluidic chip may comprise a plurality of separate microfluidic regions formed into a chip substrate, each microfluidic region configured to simulate an organ and comprising at least one inlet and at least one outlet with a microfluidic channel therebetween. The microfluidic chip may further include connecting microfluidic pathways between the separate microfluidic regions, wherein the connecting microfluidic pathways may be configured to be vascularized. The connecting microfluidic pathways may comprise a surface treatment configured to receive endothelial cells. The surface treatment may comprise an extracellular matrix protein coating. Endothelial cells may be disposed within the connecting microfluidic pathways. The endothelial cells may comprise organ-specific endothelial cells that match an organ simulated by at least one of the separate microfluidic regions. The microfluidic chip may include a first cell type of a first organ in a first microfluidic region configured for simulating the first organ, and a second cell type of a second organ in a second microfluidic region configured for simulating the second organ, wherein the first organ may be different from the second organ. The connecting microfluidic pathways may comprise SMNs having non-linear channels with physiologically relevant geometries. Alternatively, the connecting microfluidic pathways may comprise IMNs having linear channels with uniform dimensions. At least one of the separate microfluidic regions may comprise a porous wall between at least two microfluidic channels to allow molecular exchange while maintaining separation between different cell types. The microfluidic chip may further comprise at least one electrode set operably coupled with at least one of the separate microfluidic regions, wherein the electrode set may be configured to measure trans-epithelial electrical resistance of cells. The microfluidic chip may further comprise an oxygen sensor operably coupled with at least one of the separate microfluidic regions. The microfluidic chip may further comprise a camera operably coupled with at least one of the separate microfluidic regions.

The plurality of separate microfluidic regions may comprise at least one of: a brain microfluidic region configured to simulate a blood-brain barrier, a lung microfluidic region configured to simulate an alveolar-capillary barrier, a liver microfluidic region configured to simulate hepatic metabolism, a kidney microfluidic region configured to simulate a glomerular filtration barrier, a heart microfluidic region configured to simulate myocardial tissue perfusion, a gut microfluidic region configured to simulate intestinal absorption and barrier function, and many other organ-specific microfluidic regions. Each separate microfluidic region may include cells of the organ being simulated. The microfluidic chip may further comprise butterfly ports between the separate microfluidic regions, each butterfly port including at least two port lobes and a fluidic connector. The microfluidic chip may comprise at least three separate microfluidic regions that may be interconnected by the connecting microfluidic pathways that may be configured to be vascularized. The microfluidic chip may comprise at least one set of electrode recesses formed into the chip substrate adjacent to at least one separate microfluidic region.

A microfluidic system may comprise the microfluidic chip and at least one pump operably coupled with the plurality of separate microfluidic regions to cause directional fluid flow through the separate microfluidic regions and the connecting microfluidic pathways. The microfluidic system may further comprise a fluidic control layer that may be coupled with the chip substrate so as to have at least two ports fluidly coupled with each separate microfluidic region. The at least one pump may be configured to provide either unidirectional flow or recirculating flow through the microfluidic chip. The microfluidic system may further comprise at least one bubble trap configured to prevent air bubbles from entering the microfluidic chip.

A method of assaying a multi-organ response may comprise providing a microfluidic chip comprising a plurality of separate microfluidic regions configured to simulate different organs and connecting microfluidic pathways between the separate microfluidic regions, wherein the connecting microfluidic pathways may be vascularized with endothelial cells. The method may further comprise introducing a test agent into a first microfluidic region of the plurality of separate microfluidic regions, circulating fluid through the connecting microfluidic pathways to transport the test agent or cellular products from the first microfluidic region to a second microfluidic region, and monitoring a response in the second microfluidic region. Monitoring the response may comprise measuring at least one of trans-epithelial electrical resistance, oxygen consumption, cellular morphology, barrier integrity, cytokine release, cell viability; or immune cell adhesion and migration. The test agent may comprise at least one of a visual particle, a cell, an inflammatory agent, an anti-inflammatory agent, a drug, a toxin, an antigen, or an infectious agent. The method may further comprise determining a biological response of the cells in the first microfluidic region to the test agent, and determining a biological response of the cells in the second microfluidic region to the test agent or one or more analytes. The method may further comprise obtaining data from the microfluidic chip and recording the data to a non-transitory memory device. The method may further comprise controlling a flow rate and a temperature of fluid in the microfluidic chip with a computing device. The method may further comprise monitoring tissue barrier integrity and oxygen consumption during exposure to the test agent.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a diagram of an embodiment of multi-organ assay system having a fluid control layer and a multi-organ chip layer, along with a cover and electrode layer.

FIG. 2 includes a diagram that shows a two-organ chip, where each organ is enlarged to show the microfluidic features.

FIG. 3 includes a diagram of a microfluidic chip coupled to a pump, and illustrates two different ways sensors can be operably coupled with an organ tissue region.

FIG. 4A illustrates a diagram of a three organ chip where the organs are in series with regard to the fluid flow.

FIG. 4B illustrates a diagram of a three organ chip where the first organ output is split to two different organs.

FIG. 5A illustrates a diagram of a two organ chip having a SMN region between each connection line.

FIG. 5B illustrates a diagram of a two organ chip having a SMN region between each connection line.

FIG. 6 illustrates a diagram of a computing device that can be used to control the system described herein, as well as collect or analyze data from the electrodes, cameras, and/or sensors.

FIG. 7 illustrates a diagram of a two organ chip having a cascading and narrowing microvascular network between the ends of the two different organ regions.

FIG. 8A illustrates a diagram of a three organ chip where one organ includes a stick electrode system with associated electrolyte reservoirs.

FIG. 8B illustrates a diagram of a three organ chip having embedded electrodes.

FIG. 8C illustrates a diagram of a three organ chip without embedded electrodes.

FIGS. 9A-9C illustrate diagrams of a TEER on chip designs using idealized microvascular networks along with integrated electrodes and contact pads that have been micropatterned into a substrate (e.g., polymer or glass).

FIGS. 10A-10F include graphs that show levels of cytokines and chemokines measured in effluents collected from brain tissues of the multi-organ devices after inducing inflammation using cytokine mixture.

FIGS. 11A-11F include graphs that show levels of cytokines and chemokines measured in effluents collected from lung tissues of the multi-organ devices after inducing inflammation using cytokine mixture.

FIGS. 12A-12B include graphs showing data outputs for migration and adhesion assays in brain tissue.

FIG. 12C includes a graph for adhesion assay output in lung tissue of the multi-organ devices.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes a microfluidic chip that has a plurality of separate microfluidic regions formed into a chip substrate, wherein each microfluidic region may be configured to simulate an organ. Each microfluidic region may comprise at least one inlet and at least one outlet with a microfluidic channel therebetween. The microfluidic chip may further include connecting microfluidic pathways between the separate microfluidic regions, wherein the connecting microfluidic pathways may be configured to be vascularized.

In some embodiments, the connecting microfluidic pathways may comprise a surface treatment configured to receive endothelial cells. The surface treatment may comprise an extracellular matrix protein coating. Endothelial cells may be disposed within the connecting microfluidic pathways. In certain implementations, the endothelial cells may comprise organ-specific endothelial cells that match an organ simulated by at least one of the separate microfluidic regions.

The microfluidic chip may further comprise a first cell type of a first organ in a first microfluidic region configured for simulating the first organ, and a second cell type of a second organ in a second microfluidic region configured for simulating the second organ, wherein the first organ may be different from the second organ.

In some embodiments, the connecting microfluidic pathways may comprise synthetic microvascular networks (SMNs) having non-linear channels with physiologically relevant geometries. The physiologically relevant geometries may comprise bifurcations, varying cross-sectional areas, and convolutions. Alternatively, the connecting microfluidic pathways may comprise idealized microvascular networks (IMNs) having linear channels with uniform dimensions.

At least one of the separate microfluidic regions may comprise a porous wall between at least two microfluidic channels to allow molecular exchange while maintaining separation between different cell types. The microfluidic chip may further comprise at least one electrode set operably coupled with at least one of the separate microfluidic regions, wherein the electrode set may be configured to measure trans-epithelial electrical resistance of cells. An oxygen sensor may be operably coupled with at least one of the separate microfluidic regions. Additionally, a camera may be operably coupled with at least one of the separate microfluidic regions.

The plurality of separate microfluidic regions may comprise various organ-specific regions, each configured to simulate different physiological barriers or functions. For example, the microfluidic chip may include a brain microfluidic region configured to simulate a blood-brain barrier, a lung microfluidic region configured to simulate an alveolar-capillary barrier, a liver microfluidic region configured to simulate hepatic metabolism, or other organ-specific regions. Each separate microfluidic region may include cells of the corresponding organ.

The microfluidic chip may further comprise butterfly ports between the separate microfluidic regions, each butterfly port including at least two port lobes and a fluidic connector. The microfluidic chip may comprise at least three separate microfluidic regions that are interconnected by the connecting microfluidic pathways that are configured to be vascularized. Additionally, the microfluidic chip may comprise at least one set of electrode recesses formed into the chip substrate adjacent to at least one separate microfluidic region.

A microfluidic system may comprise the microfluidic chip and at least one pump operably coupled with the plurality of separate microfluidic regions to cause directional fluid flow through the separate microfluidic regions and the connecting microfluidic pathways. The system may further comprise a fluidic control layer that is coupled with the chip substrate so as to have at least two ports fluidly coupled with each separate microfluidic region. The at least one pump may be configured to provide cither unidirectional flow or recirculating flow through the microfluidic chip, which can be controlled by the fluidic control layer (e.g., selective pathway use, valves). The system may also comprise at least one bubble trap configured to prevent air bubbles from entering the microfluidic chip.

A method of assaying a multi-organ response may comprise providing a microfluidic chip having a plurality of separate microfluidic regions configured to simulate different organs and connecting microfluidic pathways between the separate microfluidic regions, wherein the connecting microfluidic pathways are vascularized with endothelial cells. The method may further include introducing a test agent into a first microfluidic region of the plurality of separate microfluidic regions, circulating fluid through the connecting microfluidic pathways to transport the test agent or cellular products from the first microfluidic region to a second microfluidic region, and monitoring a response in the second microfluidic region.

Monitoring the response may comprise measuring at least one of trans-epithelial electrical resistance, oxygen consumption, cellular morphology, barrier integrity, cytokine release, cell viability; or immune cell adhesion and migration. The test agent may comprise at least one of a visual particle, a cell, an inflammatory agent, an anti-inflammatory agent, a drug, a toxin, an antigen, or an infectious agent.

The method may further comprise determining a biological response of the cells in the first microfluidic region to the test agent, and determining a biological response of the cells in the second microfluidic region to the test agent or the one or more analytes. Additionally, the method may include obtaining data from the microfluidic chip, wherein the data comprises an image, a video, a voltage, an oxygen sensor reading, a temperature, or a flow rate, and recording the data to a non-transitory memory device. The method may also include controlling a flow rate and a temperature of fluid in the microfluidic chip with a computing device, and monitoring tissue barrier integrity and oxygen consumption during exposure to the test agent.

In some embodiments, the present microfluidic device can be used in a method for assaying multiorgan systemic inflammation and toxicity. In some aspects, the device includes two, three, or more interconnected microscale devices that are each configured to mimic an organ. These components can be connected by vascularized fluidic channels, which can be lined with endothelial cells that either match those of the adjacent organ or come from a different source. The device can include optically clear plastic microfluidic chip containing channels 100-500 microns (μm) in diameter. The flow channels can be lined with native or synthetic extracellular matrix proteins and overlaid with human or animal endothelial cells derived from specific organs. The tissue compartments can contain organ-specific human or animal cells. The apparatus can also include both biological sensor and fluidics control layers, which work together to maintain the physiological conditions required for tissue health and enable monitoring of tissue damage caused by systemic inflammation. However, the sensors can alternatively be embedded in the chip substrate or operably coupled therewith. The microfluidic device can be used for various multi-organ assays.

For example, a method can be performed that measures cytokine release and leukocyte-driven endothelial dysfunction by introduction and circulation of peripheral blood mononuclear cells (PBMCs) within whole blood. A second method screens anti-inflammatory agents delivered in circulating whole blood to assess their ability to resolve inflammation. Another method monitors cellular response to test agents, and how the cellular response causes cascading effects on the other organs in the multi-organ chip.

In some embodiments, the device be used for replicating both primary and secondary inflammatory responses, whether induced by pathogens or cytokines, across multiple organs, using human or animal cells. For example, the cells can be sourced from non-autologous donors. The configuration of the multi-organ chip also enables the screening of anti-inflammatory agents delivered in circulating whole blood to assess their ability to resolve inflammation.

The invention allows multiple organ systems to be fluidically connected on a single microfluidic chip through vascularized connector channels. This overcomes problems associated with non-vascularized connector channels. This advancement enables the development of predictive models that more closely mimic human systemic circulation, tissue physiology, inflammation, and drug responses.

In some embodiments, the system supports either unidirectional or recirculating fluid flow using an in-line pump. In some aspects, the chip can includes biological sensors to noninvasively monitor key physiological parameters such as TEER, pH, temperature, and oxygen. These features allow for real-time assessment of tissue health and early detection of damage caused by systemic inflammation.

In some embodiments, the fluidic control layer and ports associated with the organ regions as well as the vascularized interconnections allow for the co-culture of multiple cell types from different tissues. In some aspects, the different cultures can include cells from both autologous and nonautologous sources. The ability to combine different cell sources into a unified, functional unit is essential, as each source may express distinct cellular biomarkers critical for biological accuracy and disease modeling. The design also includes butterfly ports, which uniquely allow regions of the chip to be closed off during seeding, so certain regions can receive certain cells and other regions receive other cells. This enables organ-specific endothelial cells to be precisely matched to corresponding tissues.

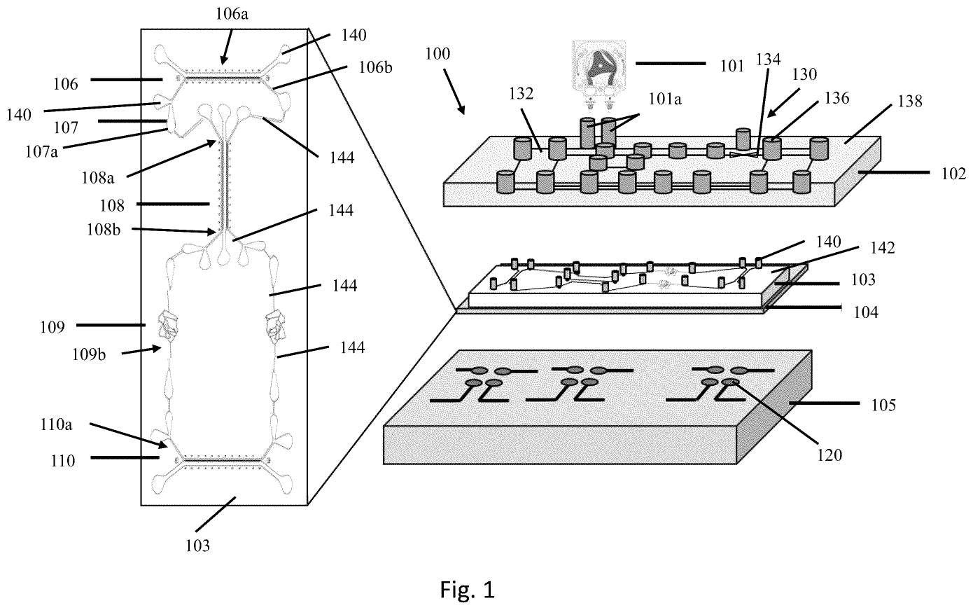

In some embodiments, the system connects multiple organ models on a chip, utilizing flow channels coated with extracellular matrix proteins and overlaid with human or animal endothelial cells. The apparatus consists of at least three interconnected microscale devices, fabricated from a single polymer block, and bonded to a glass slide (see FIG. 1). It replicates vascularized tissues from at least two distinct organs, facilitating the study of systemic injury processes, such as inflammation, drug toxicity, and infection, and their downstream effects on other organs.

FIG. 1 is a diagram of an assay system in which a solution, such as a synthetic fluid or whole blood, is either passed once or recirculated through the multiorgan microfluidic device 100 using an in-line pump 101 (e.g., either unidirectional or peristaltic, or plurality of pumps 101) connected to a fluidics control layer 102 that is configured to be fluidly coupled to a multiorgan microfluidic chip 103. A cover 104 is positioned on the multiorgan microfluidic chip 103 so as to form a surface or side of a three-dimensional microfluidic organ (e.g., microfluidics configured to simulate an organ). Additionally, a sensor layer 105 with sensors 120 formed therewith ca be provided to associate the sensors 120 with assay regions. The sensors 120 can be biological sensors, which can be integrated or otherwise coupled or associated with the sensor layer 105 enable real-time monitoring of tissue conditions, such as transendothelial electrical resistance (TEER) or oxygen levels. However, the sensor layer 105 can be omitted and the sensors can be coupled with or integrated with the chip 103 or cover 104.

FIG. 1 at the left side also illustrates the components of the multiorgan microfluidic chip 103, which can be polymer-glass multiorgan microfluidic chip that is designed using IMN and SMN architectures to perform the systemic inflammation assay. As shown, a first organ region 106 includes a butterfly port 107. The butterfly port 107 is positioned at one fluidic connection between the first organ region 106 and second organ region 108. The first organ region 106 includes two port inlets 140 at opposite sides of the assay region 106a, which has two outlets 106b that are both connected to an inlet 108a of the second organ region 108. The second organ region 18 includes two outlets 108b that are fluidly coupled to vascular networks 109. The outlets 109b of the vascular networks 109 are connected to two inlets 110a of the third organ region 110.

In FIG. 1, an in-line pump 101 is shown for systemic circulation (e.g., peristaltic pump, or other pump). The pump 101 is operably coupled to a fluidics control layer 102. The multiorgan microfluidic chip 103 is configured to be fluidly coupled with the fluidics control layer 102 (e.g., polymer layer; PDMS, SEBS, or COC), such that flow direction and flow rate is controlled at each channel and organ region. The cover 104 can be a glass slide or other transparent material. The sensor layer 105 can be configured as a biological sensor layer with integrated sensors 120 (e.g., TEER; oxygen, etc.)

The multiorgan microfluidic chip 103 includes the first organ region 106 configured as an IMN microfluidic device. The butterfly port 107 has an outlet to one of the inlets to the first organ region 106 and an outlet to the second organ region 108. The butterfly port design allows for an fluidic port from the fluidics control layer 102 into each lobe 107a, and each lobe 107a can independently have inlet flow or outlet flow, which is controlled by the pumps 101 and valves of the fluidics control layer 102. Additionally, the second organ region 108 is configured as an IMN microfluidic device, which is connected to the vascular networks 109, which are each SMNs. Then, the third organ region 110 is configured as an IMN microfluidic device.

With reference to the fluidics control layer 102, the pump 101 is connected to pump ports 101a, where two pump ports 101a are illustrated. However, any number of pumps 101 and pump ports 101a could be used, in any distribution and in any alignment. The pump ports 101a are fluidly coupled with a fluidic network 130 having a plurality of channels 132 between the two pump ports 101a and the plurality of chip ports 136. Each of the plurality of channels 132 can include a valve 134, where only one valve 134 is shown in one channel 132 to a chip port 136. However, for clarity, each channel 132 is considered to include a valve 134 to regulate flow to the respective chip port 136. Notably, the fluidics control layer can include the chip ports 136 oriented towards the multiorgan microfluidic chip 103. As such, the fluidics control layer 102 may be flipped, or the chip ports 136 may be located on the other side of the substrate 138. The channels 132 be above, within or below the substrate 132 so as to fluidly coupled with the chip ports 136.

The chip ports 136 are positioned to align with the ports 140 of the multiorgan microfluidic chip 103. While shown to be protruding or include a port nozzle, the ports 140 may also be receptacles for the chip ports 136. Accordingly, the valves 134 can be used to control flow into or out from the chip ports 136 connected to the ports 140. In some aspects, the ports 140 can include a butterfly port 107, which includes two lobes 107a. Therefore, the location of the ports 140 relative to the structure of the different organs on the multiorgan microfluidic chip 103 can be used to selectively control flow into, through, and/or out from the different organs. Moreover, the pump 101 and valves 134 can be used to tailor flow rates and perfusion through the simulated tissues in the organs on the chip 102. The control layer 102 can be controlled by a computer controller (e.g., computing system) that can execute flow control instructions to operate the pump and/or control layer.

The cover 104 can be used to cover the microchannel and organ structures formed into the chip substrate 142.

While the sensor layer 105 is shown as a separate layer, the sensors 120 can be coupled or associated with or embedded in any of the substrates or any of the layers.

However, for clarity the sensor layer 105 illustrates the sensors 120 being adjacent to the microchannels and organs on the chip 102. The sensors 120 can be any type of sensor, such as those described herein or otherwise known. The lines associated with the sensor indicates electronic leads that can be coupled with a sensor controller and/or sensor data collector (e.g., computer system).

In the chip layer 102, the first organ region 106 can be configured as a first organ type with cells indicative of that organ type. The second organ region 108 can be configured as a second organ type with cells indicative of that second organ type. The third organ region 110 can be configured as a third organ type with cells indicative of that third organ type. The vascular networks 109 can include endothelial cells so as to mimic a physiological vascular network. Since the vascular network can be considered to be an organ, the example chip 102 is shown to include four separate organ region.

Additionally, it is noted that each of the organs are connected by connecting channels 144 that connect one organ region to another organ region. The connecting channels are configured to mimic physiological blood vessels by including endothelial cells. In certain embodiments, the device comprises one or more microchannels configured to support the culture, maintenance, or growth of vascular-associated cells. The microchannels may be dimensioned and structured to mimic the geometry, flow dynamics, and surface characteristics of natural blood vessels. The microchannels may be formed of a biocompatible material and may include one or more coatings, surface modifications, or extracellular matrix components to promote cell adhesion and viability. The microchannels are configured to culture one or more vascular cell types, including but not limited to, endothelial cells, smooth muscle cells, pericytes, and fibroblasts. In some embodiments, the microchannels may support co-culture of multiple cell types to replicate the multi-layered architecture of blood vessels. The microenvironment within the microchannels may be designed to permit perfusion, shear stress, nutrient delivery, and waste removal in a manner analogous to in vivo conditions. Optionally, the device may include one or more ports, reservoirs, or membranes to facilitate fluid exchange or communication with adjacent compartments. The configuration enables the microchannels to serve as an in vitro model for vascular biology, drug testing, disease modeling, or tissue engineering applications. In some aspects, only endothelial cells are cultured in the microchannels 144.

As shown, the architecture of the vascularized flow channels may include linear or bifurcating channels, forming an IMN or a SMN. The system integrates both biological sensor and fluidics control layers, which work in tandem to maintain physiological conditions essential for tissue health while enabling the monitoring of tissue damage or changes due to the assay conditions, for example caused by systemic inflammation. In some aspects, key features of the system include fully developed, three-dimensional (3D) vascularized interconnections between organs on the chip, ensuring that circulating immune cells or test agents (e.g., therapeutics) are always in contact with endothelialized channels during circulation. Additionally, organ-to-organ connections using a butterfly port 107 design that allows channels to be opened or closed during cell seeding, enabling organ-specific endothelial cells to be seeded alongside the corresponding tissue type. This capability ensures precise endothelial-to-tissue matching within the multiorgan system, closely mimicking the human body. Also, the devices configuration with ports and controlled pumping provides options for unidirectional or recirculating fluidic systems. The flow can be powered by an in-line pump, which circulates cells, test agents, oxygen, or other materials. For example, the system can circulate cytokines and peripheral blood mononuclear cells (PBMCs) to recreate leukocyte-induced endothelial damage.

The sensors can be any type of biological sensors. The sensors can be coupled or integrated into the system at any layer to noninvasively monitor key physiological parameters, such as transendothelial electrical resistance (TEER), pH, and oxygen levels, allowing for real-time assessment of tissue health and detection of damage caused by systemic inflammation. Additionally, the sensors can include imaging sensors, or imaging sensors can be added in addition to the biological sensors described herein. Accordingly, a imaging sensor, such as a camera can record images or video of any organ region or microchannel in the microfluidic network on the chip. For example, the sensors 120 may be imaging sensors.

Additionally, the chip can include the plurality of integrated ports 140 that enable the selective introduction or collection of effluent samples from different regions of the multiorgan chip. The use of the control layer 102 can selectively provide media, gas, test agents, cells, or other material before, during or after an assay. This configuration provides flexibility in capturing biological data (e.g., images or metabolomic readouts) from the individual organ regions or vascular regions (e.g., whether organ or connector microchannel).

Additionally, the substrates of the layer can be prepared from optically transparent components. This allows for imaging from various positions with respect to the organs or connecting vasculature. Also, the glass slides or transparent covers allow high-magnification imaging. For example, the imaging can allow visual monitoring of of organ-specific morphology and toxicology responses.

### Embodiment #1 (Brain-Lung Two-Organ Microfluidic System)

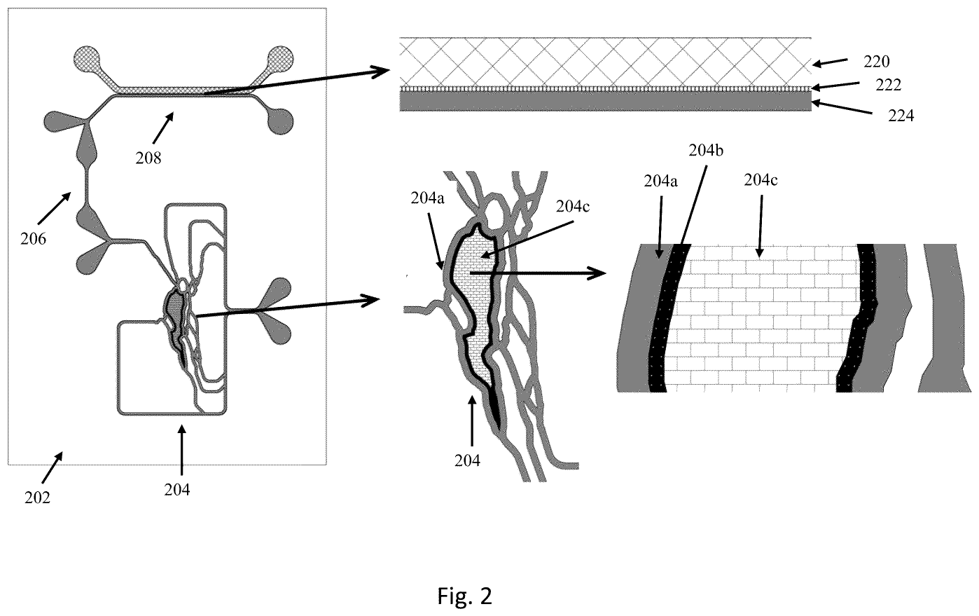

The Blood-Brain Barrier (BBB)-Lung Multiorgan System includes two interconnected tissue chips integrated in a 2-plex format (see FIG. 2). The system is designed to model the interaction between peripheral inflammation and the central nervous system with respect to the BBB. Accordingly, one organ region (202) on the chip models the BBB, such as by using a triculture of astrocytes, pericytes, and endothelial cells. The other organ region (204) models the lung, such as using a coculture of bronchial epithelial and endothelial cells.

FIG. 2 shows a diagram of a two-organ microfluidic chip 202 designed to model the brain-lung system. The similarly shaped feature of the chip 202 compared to chip 102 may be considered to be the same features. For example, the chip 202 can include a polymer-glass chip that uses IMN and SMN architectures to carry out multi organ assays, such as a systemic inflammation assay. The chip 202 includes a lung region 204 connected through a connector microchannel 206 to a blood brain barrier region 208. The blood brain barrier region includes a tissue culture region 220 and a porous barrier region 222 separating the tissue culture region 220 from a vascular culture region 224. The connector microchannel 206 can be a vascularized interconnection between the lung region 204 and the blood brain barrier region 208. The lung region 204 is enlarged to show the lung vascular region 204a, porous barrier 204b, and lung tissue chamber 204c.

The blood brain barrier region 208 can include components of an IMN microfluidic chip so simulate the BBB. The blood brain barrier region 208 includes the IMN tissue area (e.g., tissue culture region 220) for seeding brain tissue cells (e.g., astrocytes and pericytes). The porous barrier region 222 can be a prefabricated porous barrier composed of a polymer (e.g., PDMS, SEBS, or COC) and coated with native or synthetic proteins. The vascular culture region 224 includes an IMN vascular area for seeding brain endothelial cells. The connector microchannel 206 provides a vascularized interconnection between the brain and lung tissues. The lung region 204 includes the components of the SMN microfluidic chip for the vascularized lung tissue. The SMN lung tissue area (lung tissue chamber 204c) is surrounded by asymmetric and bifurcating vascular channels (lung vascular region 204a). The lung vascular region 204a provides an example of the SMN vascular area for seeding lung endothelial cells. The porous barrier 204b can be configured as an SMN prefabricated porous barrier composed of a polymer (e.g., PDMS, SEBS, or COC) and coated with native or synthetic proteins. The lung tissue chamber 204c is configured as a SMN tissue area for seeding lung epithelial cells (e.g., bronchial epithelial cells).

In some embodiments, the chip 202 can be used for various biological assays relevant to the BBB and lung. For example, after establishing continuous fluid flow through the vascular channels, an inflammatory response is selectively induced in the lung tissue using a cytokine cocktail, pathogen, or other pro-inflammatory agent. This is done by opening specific ports to expose only the lung compartment to an inflammatory stimulus, while the brain compartment receives standard media without cytokines. Following the exposure period, the lung tissue is flushed with fresh media, and the ports are rescaled to allow media recirculation between the two organ compartments.

Once the system is closed and recirculating, primary human peripheral blood mononuclear cells (PBMCs) in whole blood are introduced into the vascular channel to simulate immune system activity. This setup allows for the assessment of how lung-specific inflammation and circulating immune cells impact BBB integrity.

To monitor tissue health and function in real time, the chip can incorporate embedded gold electrodes beneath the BBB tissue to measure transendothelial electrical resistance (TEER), which reflects barrier integrity and potential immune-mediated damage. A fiber-optic microsensor can also be embedded in the lung tissue to track dissolved oxygen levels as a readout of lung tissue viability and metabolic activity.

The IMN BBB device features two parallel microchannels: tissue culture region 220 can be an apical channel (500 μm wide, 100 μm high) for culturing primary human astrocytes and pericytes, and vascular culture region 224 can be a basal vascular channel (200 μm wide, 100 μm high) lined with endothelial cells. These are separated by 3-μm-wide wall with microfabricated slits that allow diffusion of signaling molecules and tight junction formation, mimicking the in vivo BBB environment.

The SMN lung chip contains a central tissue region (lung tissue chamber 204c) surrounded by a synthetic vascular network (200 μm wide channels), such as the lung vascular region 204a. The network design can be based on real microvascular structures and can be created using AutoCAD. A micro-pillar array can be used to support the formation of 3D bronchial epithelium (100 μm high), which is seeded from an access port in the top layer. The top layer can be a optically clear top layer that enables placement of fiber-optic microsensors for oxygen monitoring or use of imaging sensors. Vascular and tissue regions can be connected via 8-μm-wide leaky gaps using the barrier wall as described herein. The porous barrier wall allows exchange of fluid and materials, such as neutrophil migration from blood vessels into lung tissue in response to inflammatory signals.

### Embodiment #2 (Brain-Lung-Intestine Three-Organ Microfluidic System)

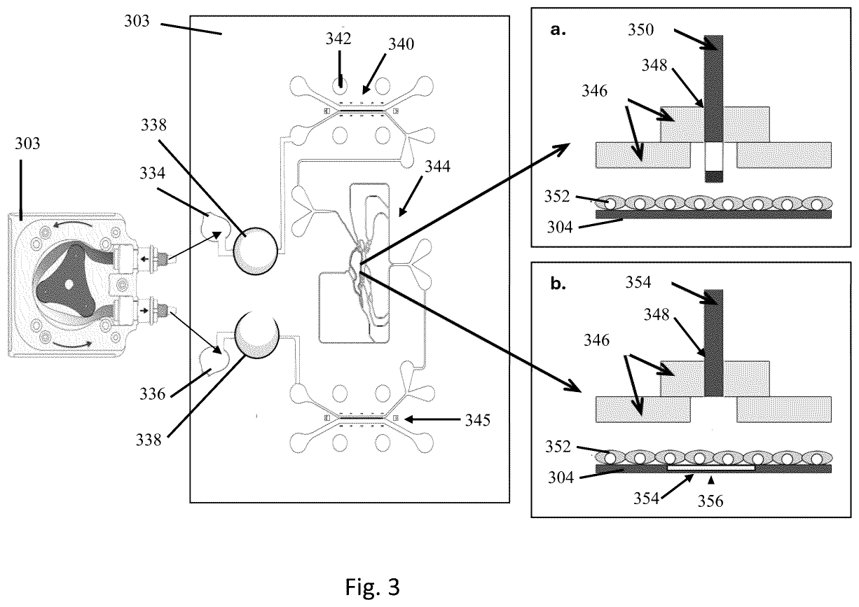

In some embodiments, the chip can be configured for flow in one direction, or multi directions, such as with unidirectional and circulating fluidics. FIG. 3 illustrates a chip 303 that is operably coupled to a pump 301. The inlet 334 and outlet 336 (e.g., which can be reversed) from the pump 301 are at fluid reservoirs 338 (e.g., gas, such as oxygen or liquid, analyte) that condition the media being pumped into the chip 303. In one example, the fluid reservoirs 338 are configured as bubble traps. The inlet 334 feeds to a first organ region 340 that is associated with electrode pads 342, which are configured as embedded electrode pads made of materials (e.g., gold, platinum, or titanium) to measure TEER. The first organ region 340 is shown to be configured as a blood-brain-barrier (BBB) as an IMN, where the biological data is obtained with TEER. The first organ region 340 is shown to feed into a second organ 344, which is configured as a lung as a SMN. The lung can be assayed as shown in panels a. and b. in FIG. 3. The second organ region 344 is shown to feed into a third organ region 345, which is then coupled through the outlet 336 to the pump 101.

In panel a., the chip substrate 303 is configured with layers 346 and having an aperture 348 with a fiber optic 350 positioned in the aperture 349. Additionally, the cover 304 can include the lung cells 352 growing thereon, and also on the chip substrate. This shows an external sensor, such as fiber optic sensor (e.g., imaging sensor, oxygen sensor, etc.).

In some embodiments, the fiber optic 350 is configured to operate in conjunction with one or more biological sensors for real-time monitoring of physiological or biochemical parameters. The biological sensors may include, but are not limited to, oxygen sensors employing luminescence quenching or absorption-based detection of oxygen concentration; pH sensors incorporating optically responsive pH-sensitive dyes; glucose sensors utilizing enzymatic detection mechanisms such as glucose oxidase with subsequent optical readout; temperature sensors comprising thermoresponsive fluorescent or phosphorescent materials; pressure sensors using interferometric or strain-sensitive optical coatings; and ion-selective sensors incorporating fluorescent ionophores for ions such as potassium, calcium, sodium, or chloride. Additionally, the fiber optic 350 may be interfaced with biomolecule sensors capable of detecting proteins, antibodies, or other analytes using techniques such as surface plasmon resonance, evanescent wave excitation, or Förster resonance energy transfer (FRET). Further, the fiber optic 350 may be used in conjunction with carbon dioxide sensors employing pH-sensitive dye systems responsive to carbonic acid concentration, and sensors for detecting microbial or cellular activity based on metabolic indicators, viability dyes, or labeled molecular probes. The integration of the fiber optic 350 with any of these sensor types enables spatially resolved, minimally invasive, and multiplexed detection of biologically relevant conditions.

In panel b., the chip substrate is configured with layers 346 and having an aperture 348 with tubing 354 (e.g., Tygon tubing, which can be coupled to the pump or other microfluidic network or fluidic control layer port, which could also be a plug). The cover 304 is shown with a data acquisition feature 354, which can be either a window or aperture, or the like. A sensor 356 can be integrated or otherwise coupled with the cover 304 at the data acquisition feature 354. Also, cover-side based data acquisitions can also be made. Thus, either the substrate or the cover can be coupled to or integrated with sensors or sensor equipment. Also, these can be internal sensors or external sensors. Here, tubing 354 can be substituted with a sensor, such as a fiber optic or other optical sensor. Also, the tubing 354 can be substituted with a plug. Also, the tubing 354 could be attached to a pump for fluid flow control. The tubing 354 can also receive instruments therethrough, such as the sensors or optical sensors described herein.

In some embodiments, a Transendothelial Electrical Resistance (TEER) sensor can be used for resistance measurements. In some embodiments, an oxygen sensor can be included into the multiorgan chip design. In some aspects, these sensors can be used to study leukocyte-mediated endothelial injury (see FIG. 3). In some aspects, unidirectional fluid flow supports the controlled seeding and maintenance of tissue-specific cell types within the multiorgan chips. Also, circulating fluid uses can replicate physiological blood flow, allowing dynamic interactions between tissues, and circulating factors, such as inflammatory mediators released during a primary inflammatory event. The sensors provide a noninvasive, real-time measure of endothelial barrier function, which is critical for evaluating vascular integrity during inflammatory responses. The fiber-optic microsensors, spot sensors, or any other sensor or sensor equipment can be integrated into the any organ (e.g., shown here as lung, but can be other organ) compartment in order to monitor data during the assay. For the example shown in FIG. 3, dissolved oxygen levels can be measured at the lung region to ensure that oxygen levels remain stable during inflammatory challenge and confirm that any downstream effects observed in connected tissues are not due to hypoxic conditions (see, FIG. 3 panel a and panel b).

FIG. 3 shows the placement of an in-line pump (either unidirectional or peristaltic), bubble traps, and integrated sensors for monitoring TEER, and pH and/or oxygen levels in a 2-organ system. Oxygen monitoring can occur through placement of an external fiber optic oxygen sensor (panel a) or through an integrated sensor placed on the glass slide of the multiorgan device (panel b). The pump can be an in-line pump (either peristaltic or unidirectional). The fluid reservoirs can be configured as a bubble trap that also acts as a reservoir for oxygenated media. The electrode pads can be made of materials (e.g., gold, platinum, or titanium) to measure TEER. The chip substrate can be one or more layers of a polymer (e.g., PDMS, SEBS, or COC)) to form the microfluidic device. The fiber optic oxygen sensor can be threaded through a punch in both polymer layers of the chip. An integrated sensor spot can be included on the cover for oxygen or pH quantification.

The electrodes described herein may be composed of a variety of electrically conductive materials, including, but not limited to, metals, metal oxides, conductive polymers, carbon-based materials, and combinations thereof. In certain embodiments, the electrodes are formed from optically transparent conducting materials, which are advantageous for applications requiring optical access through the electrode, such as optical sensing, imaging, display technologies, and photoelectrochemical systems. Suitable optically transparent conducting materials include transparent conducting oxides (TCOs) such as indium tin oxide (ITO), fluorine-doped tin oxide (FTO), aluminum-doped zinc oxide (AZO), and gallium-doped zinc oxide (GZO), each of which offers a balance of high optical transparency in the visible spectrum and low sheet resistance for efficient electrical conductivity. The electrodes may be fabricated as transparent conducting films, which can be deposited using methods such as sputtering, chemical vapor deposition, or solution-based processing techniques onto glass, polymeric, or flexible substrates. These films may be patterned using photolithography or laser ablation to define specific electrode geometries. In other embodiments, conductive polymers such as poly(3,4-ethylenedioxythiophene) polystyrene sulfonate (PEDOT:PSS) or polyaniline can be used, offering mechanical flexibility and solution-processability, making them suitable for wearable or stretchable devices. Carbon-based materials, including graphene, carbon nanotubes, and carbon black, may also be employed either alone or in composite formulations, providing excellent conductivity, chemical stability, and in some cases, transparency when appropriately patterned or dispersed. The selection of electrode composition can be tailored based on the intended application, such as maximizing transparency for optical access, flexibility for conformal contact, or chemical resistance for harsh environments. In some embodiments, a multilayer structure may be employed, wherein a transparent conductive film serves as a base layer and a metal grid or nanowire network is overlaid to further enhance conductivity without significantly compromising transparency.

The system includes ports in the chip that have been designed to incorporate either unidirectional fluid flow or circulating fluid flow depending on the needs of the user during experimental set-up or execution of the assays. The flow can be controlled by the fluidics control layer.

In an example, during model establishment of the brain and lung tissues and before exposure to pathogens/cytokines/agents, the ports of the chip can accommodate a unidirectional syringe pump needed for seeding tissue-specific cell types into the individual channels of each tissue chip (see, FIG. 3). The secondary ports are closed off to prevent cells from leaking into other tissue chips of the interconnected chip design. After tissue establishment and exposure to pathogens/cytokines/agents, the interconnected multiorgan system can be fluidically connected in-line to the pump (e.g., unidirectional, or peristaltic pump) to achieve circulating, low flow rates required by the microvasculature. Specific secondary ports are closed off (e.g., by control layer) to guide the fluid flow through each tissue from the lung tissue (SMN device), through the brain tissue (IMN device), using the pump.

To enable TEER measurements across the brain tissue, TEER electrodes can be micropatterned onto the substrate or cover of each chip (see, FIG. 3). In the vascular channels of the brain device, two electrodes can be used to input a current signal and two electrodes can be used detect the voltage signal across a cell layer. The electrode pairs are shown to be across from each other, each associated with a different fluidic region of the IMN separated by the porous wall. The commercially available SynVivo Cell Impedance Analyzer system (SynVivo SKU: 304001) can be adapted for TEER on-chip measurements.

To ensure that oxygen levels remain stable during inflammatory challenge and confirm that any downstream effects observed in connected tissues are not due to hypoxic conditions, oxygen sensors can be incorporated into the multiorgan chip. Two on-chip design options for oxygen sensor placement can be used (see, FIG. 3 panel a and panel b), or at same time if desired. One on-chip design option can facilitate the use of commercial off the shelf (COTS) optical-based micro-oxygen sensors. An external optic microprobe can be threaded through tubing in a top punch over the SMN device and introduced into the lung tissue to read dissolved oxygen levels (FIG. 3 panel a). Functionalized and non-functionalized chips can be measured for baseline oxygen levels prior to treatment. A second on-chip design option incorporates a contactless sensing foil placed in the lung tissue area (FIG. 3 panel b), which can provide an on-chip alternative to the microsensor insertion based on the sensitivity and resolution required for accurate readouts.

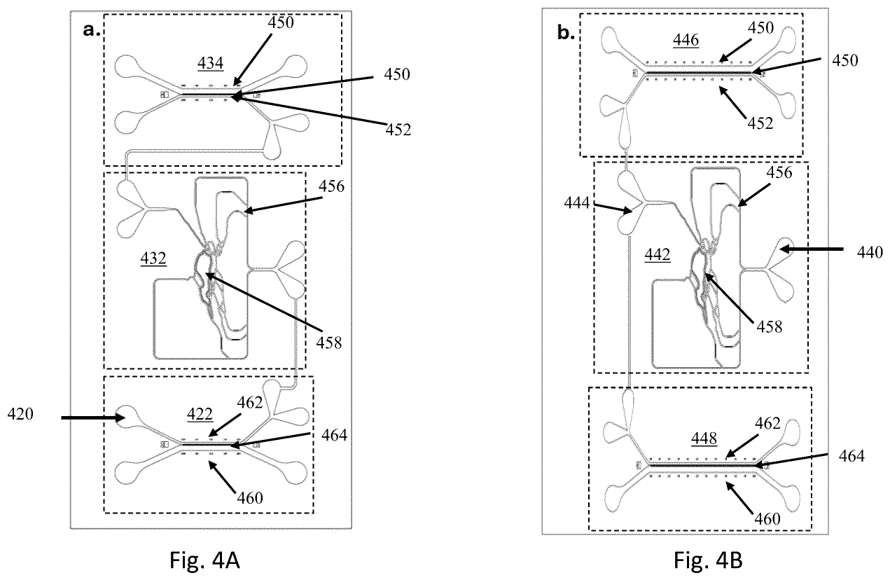

The Blood-Brain-Barrier (BBB)-Lung-Intestine Multiorgan System can include three tissue chips (e.g., three separate tissue regions) within a 3-plex format (see FIGS. 4A-4B). Each device can be used to establish and characterize one of three human primary tissues: (1) astrocytes-pericytes-blood vessel triculture for a blood-brain barrier (BBB) model; (2) normal bronchial epithelium-blood vessel coculture for a lung model; and (3) intestinal epithelium-blood vessel coculture for a gut model. Embedded gold electrodes can be patterned onto the glass slide of the chip to measure TEER in the BBB and intestinal tissues, and a fiber optical microsensor can be threaded into the lung tissue to read dissolved oxygen levels. The chips can be made from polymers (PDMS, SEBS, or COC) and a glass slide.

FIGS. 4A-4B shows diagrams of three-organ microfluidic chips that are designed to model the brain-lung-intestine system, which is similar as shown in FIG. 3 but with different flow connections and route. For example, different assays may use different flow connections and routes. For example, the illustrated polymer-glass chip 403 in FIGS. 4A04B uses IMN and SMN architectures to carry out a systemic inflammation assay. The vasculature is designed to provide two options for fluid flow and dissemination throughout the three organs. Design option 1 (see, FIG. 4A) flows fluid through the vasculature of each organ in a sequential manner (e.g., intestine-lung-brain). Design option 2 (FIG. 4B) flows fluid through the vasculature of one organ (SMN device), and the fluid path is subsequently split into two streams that sustain the other two organs (e.g., lung-brain and lung-intestine).

In FIG. 4A, the test fluid is introduced at the inlet 420 into vasculature, and then perfuses through vasculature of the first organ 422 (e.g., IMN device) of the 3-organ system. Then, the fluid perfuses sequentially through vasculature of the second organ 432 from the first organ 422. Then, the fluid perfuses sequentially through vasculature of the third organ 434 (IMN device) from the second organ 432. Notably, the organ to organ connections are vascularized, and include endothelial cells.

In FIG. 4B, the test fluid is introduced at the inlet 440 into vasculature, and then perfuses through vasculature of the first organ 442 (SMN device) of a 3-organ system. The outlet 444 of the first organ 442 is configured as a butterfly port (444) design that splits the fluid stream for equal distribution to the other two organs in 3-organ system. The butterfly port 444 provides fluid that perfuses through the vasculature of the second organ 446 (IMN device) and through the vasculature of the third organ 448 (IMN device).

In some embodiments, the top organ region (e.g., 434 and 446) can be configured as a 2-channel BBB device that includes an apical channel 450 for seeding primary human astrocytes and pericytes (e.g., 500 μm wide), which is separated from the vascular channel 452 (e.g., 200 μm wide) by a porous wall 454 having a series of 3-micron microfabricated interstitial channels or slits. All channels are 100 μm high. The slits allow for diffusion gradients to form and biomolecular crosstalk to occur between cell types of the BBB, which contributes to barrier tightness that simulates in vivo BBB barrier function.

In some embodiments, as shown the SMN is configured as a lung device that includes a synthetic vascular network 456 (e.g., 200 μm wide) surrounding a central tissue site 458 where bronchial epithelial cells can be seeded. The architecture and bifurcation angles for the synthetic vascular network were rendered in AutoCAD from in vivo images of microvascular network structures, creating a realistic simulation of vascular network dynamics surrounding the lung tissue. A micro-pillar array can be used in the central tissue site 458 used to create scaffolds to support a 3D lung tissue (e.g., 100 μm high). Bronchial epithelial cells are seeded from a hole punched directly on top of the tissue area and sealed with an optically clear layer of polymer with tubing (e.g., see FIG. 3 panel b). In addition to facilitating the seeding of bronchial epithelial cells, the top layer provides direct tissue access for fiber optic microsensors (FIG. 3 panel a). Leaky gaps (8 μm wide) are used to connect the vascular channels with the tissue area using a walled barrier method. The gaps are wide enough to accommodate leukocyte trafficking from the vasculature into the lung tissue in response to inflammatory signals.

In some embodiments, the bottom organ (e.g., 422 and 448) can be configured with side-by-side architecture as a 2-channel intestine device includes an apical chamber 460 with intestinal epithelial cells (e.g., 500 μm wide) that is separated from a vascular channel 462 (e.g., 200 μm wide) by a porous wall 464 having a series of microfabricated interstitial channels or slits. The slits are 3-5 microns wide, which allows for diffusion gradients to form and biomolecular crosstalk to occur between cell types. All channels are 100 μm high, which creates a small tubular intestinal vessel or vasculature to achieve the physiological structure of the gut including scale, morphology, and vascularization.

### Embodiment #3 (Lung-Immune-Neuro Axis [LINA] Two-Organ or Three-Organ Microfluidic System) for Air-Liquid Interface (ALI)-Based Studies

Neurotropic agents and respiratory viruses have been associated with strong resident and systemic inflammatory responses that disrupt both the respiratory tract and the BBB, which in turn contribute to organ damage and degenerative manifestations. Accordingly, the multiorgan microfluidic chip can be configured to study systemic inflammation to various organs, such as the BBB, lung, gut, or the like.

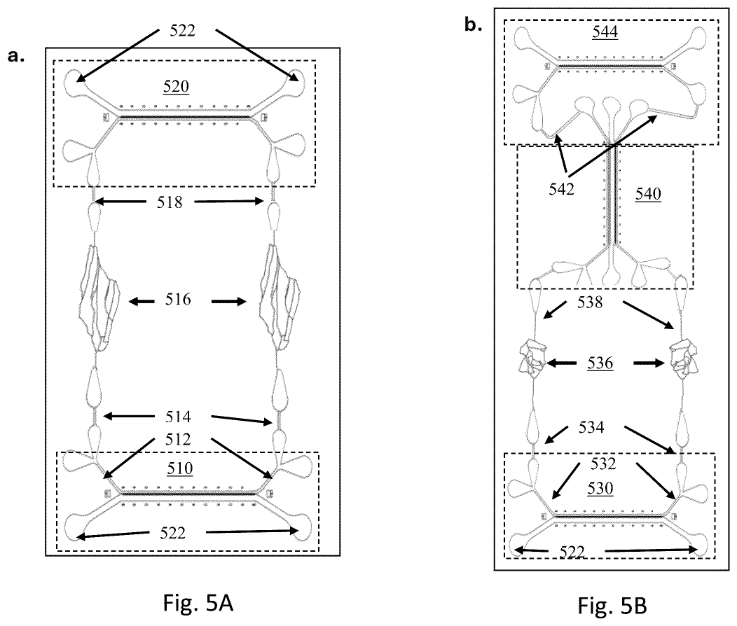

FIG. 5 shows a diagram of a two-organ plus vasculature (FIG. 5A), or three-organ plus vasculature (FIG. 5B), microfluidic chip designed to model the brain-circulatory vasculature-lung or brain-circulatory vasculature-lung-intestine system with the incorporation of an air-liquid interface (ALI) within the lung tissue. This polymer-glass chip uses IMN (tissues) and SMN (circulatory vasculature) architectures to carry out a systemic inflammation assay.

FIG. 5A shows the first organ 510 having two fluidic connections 512 through separate vascularized channels 514 through vasculature regions 516 through further separate vascularized channels 518 to two fluidic connections to the second organ 520. This shows the components of an IMN microfluidic chip for the lung tissue (e.g., first organ 510 or second organ 512), which can be placed under an air-liquid interface. Also, the vasculature regions 516 can be configured as SMN microvascular connections between tissues. This also shows the components of an IMN microfluidic chip for the brain (BBB), which can be the second organ 520 or first organ 510. The various ports 522 can be coupled with the fluidics control layer and to ports thereof so that the fluid flow through the device can be controlled. Applying flow at certain ports 522, holding pressure at other ports 522, and/or drawing fluid from certain ports 522, all at defined rates, can be used to accurately control the fluid flow and perfusion through the device and simulated organ regions.

In some embodiments, an air-liquid interface (ALI) is established within a microfluidic device to support the differentiation and physiological function of lung epithelial cells. The ALI is implemented by configuring the device to include a vascular channel and a tissue channel, wherein the vascular channel is perfused with a liquid culture medium and the tissue channel is exposed to a flow of humidified air. The tissue channel is positioned above a porous membrane that separates it from the vascular channel, thereby allowing the epithelial cells cultured on the membrane to receive nutrients from the basal side while being exposed to air on the apical side, effectively mimicking the in vivo environment of the lung. The humidified air flow is delivered to the tissue channel via an off-chip air pump system connected to a bubble humidifier, which ensures that the air entering the device is adequately humidified to prevent desiccation of the cultured cells. The flow rate and humidity levels may be controlled to replicate physiological conditions found in the human respiratory tract. This configuration promotes the differentiation of airway epithelial cells into a pseudostratified structure with functional characteristics such as ciliation and mucus production. The ALI system is particularly advantageous for modeling pulmonary biology, respiratory infections, drug delivery, and toxicology screening in a dynamic and physiologically relevant context.

FIG. 5B shows the first organ 530 having two fluidic connections 532 through separate vascularized channels 534 through vasculature regions 536 through further separate vascularized channels 538 to two fluidic connections to the second organ 540. The second organ 540 includes two flow paths, each connected with a different vascular region, and each has an outlet through further separate vascularized channels 542 to a third organ 544. This shows the components of an IMN microfluidic chip for the intestine tissue (e.g., first organ 530), which can be placed under an air-liquid interface. Also, the vasculature regions 536 can be configured as SMN microvascular connections between tissues. The second organ 540 can be a lung organ region, configured here as an IMN. This also shows the components of an IMN microfluidic chip for the brain (e.g., BBB), which can be the third organ 544. The various ports 522 can be coupled with the fluidics control layer and to ports thereof so that the fluid flow through the device can be controlled. Applying flow at certain ports 522, holding pressure at other ports 522, and/or drawing fluid from certain ports 522, all at defined rates, can be used to accurately control the fluid flow and perfusion through the device and simulated organ regions.

In this context, the two-organ or three-organ microfluidic system with microvasculature mimicking the can simulate the lung-immune-neuro axis (LINA; FIG. 5A) or intestine-lung-immune-neuro axis by connecting two or three IMN models for the BBB (IMN), lung (IMN), and intestine (IMN) with the SMN-style microvasculature to represent the systemic circulation (FIG. 5B). These interconnected chips in one platform can form a lung-brain axis with an integrated circulatory system, with the option of placing the lung tissues under an air-liquid interface (ALI). Incorporation of tissue-resident macrophages or microglia, as well as circulating immune cells (e.g., macrophages, neutrophils, and dendritic cells), can effectively simulate inflammation and infection in localized and disseminated forms. Inclusion of on-chip TEER and oxygen sensors can monitor real-time changes in tissue barrier integrity and oxygen consumption during exposure to nonreplicating or replicating insults and complete the features of the platform.

### Embodiment #4 (Heart-Skeletal Muscle Microfluidic System)

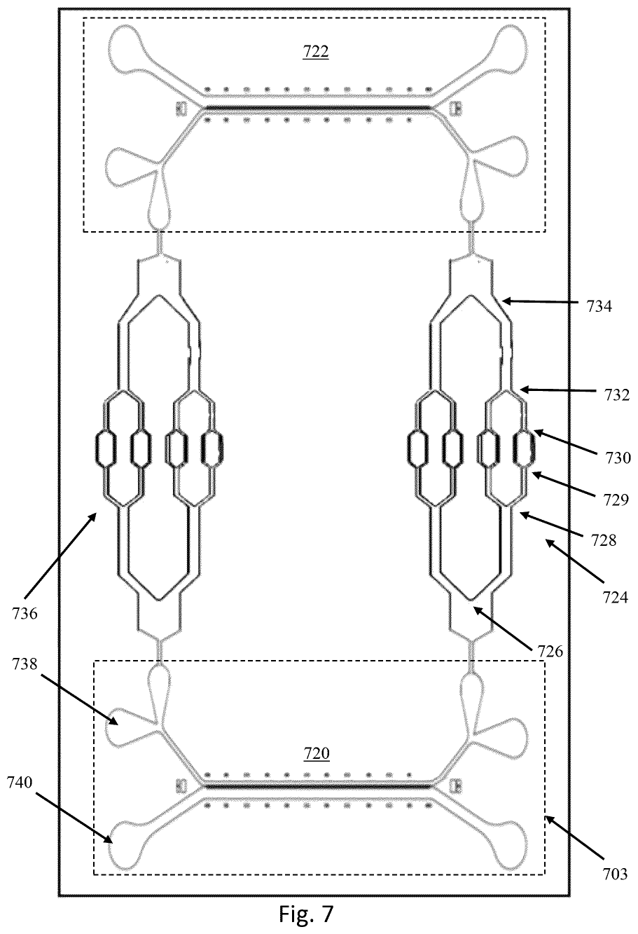

The Heart-Skeletal Muscle multiorgan chip can connect two IMN-based microphysiological models representing the human heart and skeletal muscle, integrated with vasculature in a single microfluidic platform (FIG. 7). These two organ compartments can be separated by a custom-designed microvascular bed featuring a series of progressively narrowing bifurcations. The microvascular beds get narrower towards the middle, which may impact a thrombosis that can occur. Thus, the thrombosis can be modeled. However, the design can be implemented with any number of organ regions.

FIG. 7 a chip 703 that includes a first organ region 720 separated from a second organ region 722 by a first cascading microvascular network 724 that includes at least a first narrowing bifurcation 726, second narrowing bifurcation 728, and third narrowing bifurcation 729, which is followed by a first widening junction 730, second widening junction 732, and third widening junction 734. A second cascading microvascular network 736 extends from the second organ region 722 back to the first organ region 720. Accordingly, these features simulate a vascular network, such as a capillary bed and the venules and areoles associated therewith.

For example, a sample can be introduced into a first port 740 and pass through the first organ region 720 in conditions that can induce clotting. The flow is then through the first cascading microvascular network 724 through the second organ region 722 and then through the second cascading microvascular network 736. The flow can be withdrawn at the butterfly port 738 or circulated back through the first organ region 720. This shows two tissues connected by circulation through narrowing interconnections between the tissues, which simulates narrowing of vascular beds and allows for monitoring for thrombosis.

FIG. 7 shows a diagram of a two-organ microfluidic chip designed to model the heart-circulatory vasculature-skeletal muscle system with the incorporation of vascular beds that can generate thrombosis. This polymer-glass chip uses an IMN (tissues and circulatory vasculature) architecture to carry out a clotting assay with platelets in plasma. The figure shows the components of an IMN microfluidic chip for the heart tissue comprised of cardiac smooth muscle cells and aortic endothelial cells. Also, the IMN microvascular connections can be positioned between tissues that bifurcate to form increasingly narrower channels. The components of an IMN microfluidic chip for the skeletal muscle can include skeletal muscle cells and microvascular endothelial cells.

This vascular network mimics physiological blood flow from the heart to peripheral muscle tissue and is engineered to promote thrombosis formation, simulating vascular obstruction in distal extremities such as the leg. The platform can be further enhanced by incorporating circulating immune cells (e.g., neutrophils, monocytes, and platelets) to model thromboinflammatory processes. Real-time monitoring capabilities, such as embedded oxygen and flow sensors, can enable dynamic assessment of perfusion, tissue health, and thrombus development across the heart-muscle axis.

### Embodiment #5 (Blood-Brain Barrier (BBB)-Lung-Intestine Microfluidic System with TEER Device)

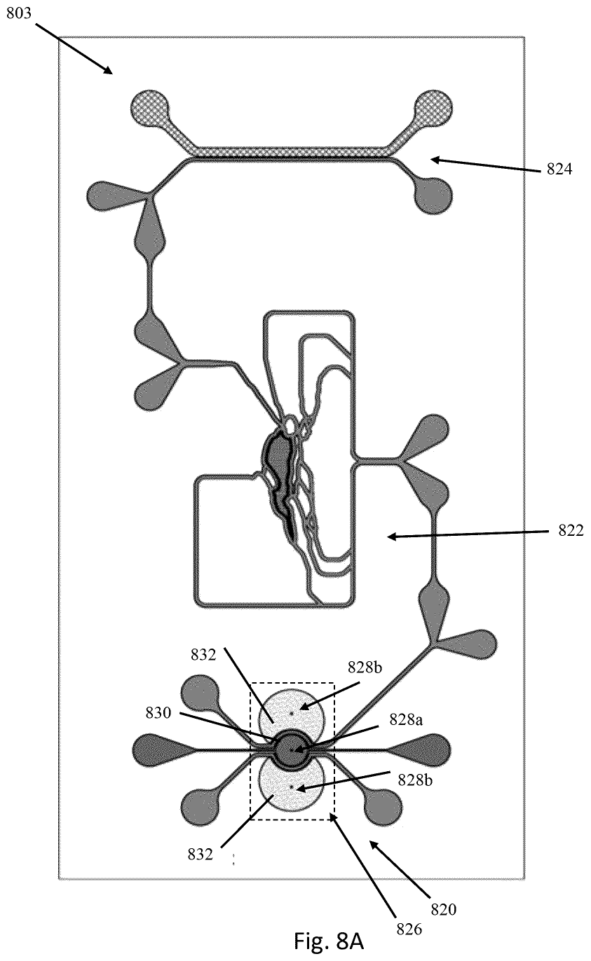

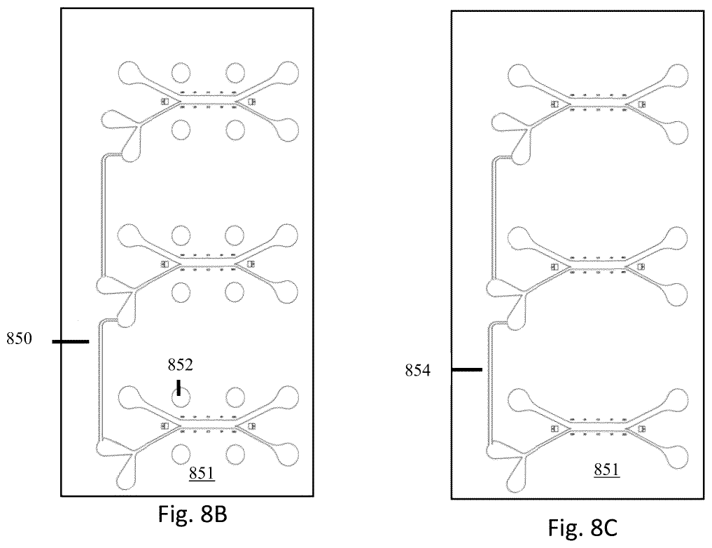

The multiorgan platform can include three interconnected tissue chips arranged in a 3-plex format (see FIG. 8A), each supporting the establishment and maintenance of distinct human primary tissue models: (1) a blood-brain barrier (BBB) model composed of astrocytes, pericytes, and endothelial cells in triculture; (2) a lung model consisting of a normal bronchial epithelium-endothelium coculture; and (3) a gut model comprising intestinal epithelium-endothelium coculture. Transendothelial electrical resistance (TEER) measurements in the BBB microfluidic chip can be enabled via two external stick electrodes made from conductive materials, such as silver chloride, gold, platinum, or titanium, which can be inserted into dedicated access ports in the microfluidic device. This setup allows non-invasive, real-time monitoring of barrier integrity.

FIG. 8A illustrates a chip 803 having a first organ region 820, second organ region 822, and a third organ region 824. The first organ region 820 includes an area 826 that is measured with an electrode system that uses stick electrodes 828a,b. The stick electrodes 828a,b include a center stick electrode 828a associated with a tissue culture region 830 and lateral stick electrodes 828b associated with electrolyte chambers 832 that are around or lateral from the tissue culture region 830. Accordingly, the systems can include electrodes without fabricating locations in the chip substrate for the electrodes. Other electrode arrangements can be used that are associated with the chip 803 instead of being fabricated into the chip 803.

FIG. 8A shows a diagram of a three-organ microfluidic chip designed to model three tissues with the added capabilities of reading TEER using external electrodes. This polymer-glass chip uses both IMN and SMN architectures. The figure shows that components of an IMN microfluidic chip for brain (BBB) tissue that incorporates TEER-based measurements using external stick electrodes made of a conductive material (e.g., silver chloride, gold, platinum, or titanium).

### Embodiment #6 (Multiplexed Blood-Brain Barrier (BBB) Microfluidic System with or without TEER Capabilities)

A Blood-Brain Barrier (BBB) multiplexed microfluidic system includes three interconnected tissue chips arranged in a 3-plex format (see FIGS. 8B-8C), each supporting the establishment and maintenance of one single tissue (e.g., BBB) in triplicate, or three single tissues. For the TEER-enabled devices, TEER electrodes can be micropatterned onto the glass slide of each chip. In the vascular channels of each chip, two electrodes can input a current signal and two electrodes can detect the voltage signal across a cell layer. This setup allows non-invasive, real-time monitoring of barrier integrity.

FIG. 8B shows an example of a three-organ microfluidic chip 850 designed to model three tissues, where the tissue regions 851 can be of any type, which are shown as IMNs. For example, one, two or three of the tissue regions can be a BBB region, or a BBB region in combination with two other organs. The connector channels between the tissue regions (e.g., organ regions) can be vascularized as described herein. FIG. 8B shows the chip 850 includes embedded electrodes 852. The chip 850 can include recesses adapted to receive the embedded electrodes 852. On the other hand, chip 854 of FIG. 8C is devoid of embedded electrodes, and does not have recesses adapted to receive electrodes. However, any of the other layers described herein could include recesses and embedded electrodes.

FIG. 3 shows a diagram of a multi organ microfluidic chip designed to model three tissues with the added capabilities of reading TEER using embedded electrodes 342. However, FIG. 4 shows the chip without the embedded electrodes. As such, the chips can be configured fabricated spaces that can receive embedded TEER electrodes, or the chips can be devoid of such fabricated spaces and be without the TEER-enabled capabilities. However, the electrodes can be associated with the chip without being fabricated into the chip.

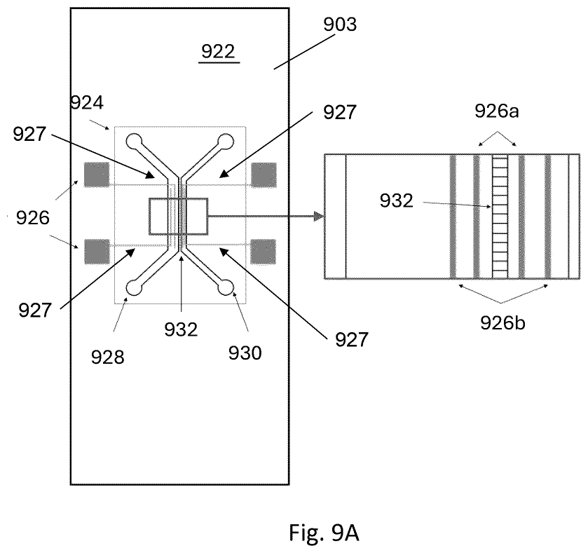

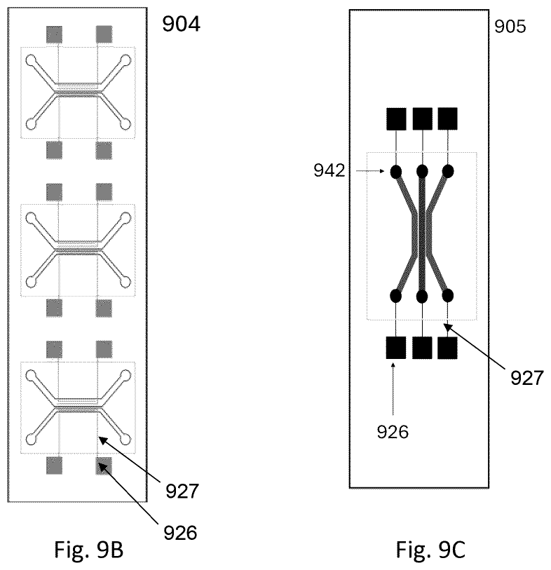

FIG. 9A illustrates a chip 903 for TEER measurement composed of a single plex configuration. The integrated device includes two layers-a PDMS fluidic (or chip) layer 924 and an electrode layer 922 with electrode contact pads 926 micropatterned onto the borosilicate glass of the electrode layer 922. The electrodes 927 connected to the electrode contact pads 926 can be made from any conductive materials including but not limited to silver chloride, gold, platinum, or titanium. The fluidic layer design can have two linear side-by-side channels—a vascular channel 928 and a tissue channel 930—separated by a perforated barrier 932. The micropatterned electrode layer can have four electrodes 927 with four electrode contact pads 926 extending beyond the edge of the PDMS. FIG. 9A also shows a close-up of the electrodes 927 on opposite sides of the barrier: two voltage electrodes 926a closest to the barrier 932), and two current electrodes (926b furthest from the barrier 932).

In the chip layer 924, the tissue channel 930 can be configured with a host of cell types including but not limited to astrocytes, pericytes, hepatocytes, tumor cells, fibroblast, smooth muscle cells, cardiomyocytes, neurons, airway epithelial cells, immortalized cell lines, primary cells and ISPC cells. The vascular channel 928 can include endothelial cells so as to mimic a physiological vascular network. In some embodiments the vascular and tissue channels can be interchanged with the tissue channel configured with endothelial cells so as to mimic a physiological vascular network and the vascular channel containing cell indicative of an organ type.

FIG. 9B illustrates a chip 904 for TEER measurements in a multiplex (3 plex configuration).

FIG. 9C illustrates a three-channel chip 905 for TEER measurements. The configuration of the chip 905 includes two tissue channels and one vascular channel or two vascular channels and one tissue channel. The chip 905 also contains embedded contact pads 926 that extend beyond the layer of PDMS and electrodes 927 that come in contact with the ports 942 of the chip 905.

In electrode layers of the chips 903, 904, 905, the lines associated with the sensors (e.g., contact pads and electrodes) indicates electronic leads that can be coupled with a sensor controller and/or sensor data collector (e.g., computer system). Further, in chip 905, electrodes may be embedded under the ports of the chip layer. This configuration can be incorporated into any electrode layer and chip design in order to avoid having electrodes impact the visualization of cells in the tissue and vascular compartment and channels.

In some embodiments, a Brain-Lung-Heat embodiment can be provided, where the multiplex microfluidic system contains three unconnected 2-channel chips (see FIG. 9B). This system is designed to evaluate a compound or compounds in three modeled systems (e.g., Brain, Lung, Heart) concurrently in a single experiment. Accordingly, one organ system on the chip models BBB using a coculture of astrocytes, pericytes in on channel, and brain endothelial cells in the other channel. Another organ system can be configured to model the lung, such as using a coculture of bronchial epithelial and lung endothelial cells. Another organ system models the heart using cardiomyocytes in one channel and aortic endothelial cell in the other channel. TEER measurements are recorded to establish barrier functionality and tissue integrity prior to administration of a reagent or compound, including but not limited to a therapeutic drug, experimental compound, toxin, proinflammatory molecule, nanoparticle, or other. TEER measurements are recorded after administration of the compound to assess the effect the compounds have on barrier functionality and tissue integrity and effluent are collected from the three organ multiplex microfluidic systems for further analysis.

In some embodiments, the multiorgan chips described herein can be used in assay methods related to the specific organs. As such, a method of using the chip described herein for an assay involving multiple organ regions. The method includes: treating vasculature associated with organ region with media alone or media plus cocktail; flowing media alone through second organ region; maintaining segregation of organ regions on the chip; rinse first organ region with fresh media; connecting first and second organ regions (e.g., with fluidic control layer 102) and flow fresh media therethrough to mimic event; obtain traceable test agent; introduce traceable test agent to test solution; and monitor the traceable test agent over time and through the chip at block.

The chip can be used for introduction and circulation of peripheral blood mononuclear cells (PBMCs) within whole blood. An example method can includes: treating vasculature associated with lung tissue region with media alone or media plus cytokine cocktail under flow for a period of time (e.g., 4 hours); flowing media without cytokine cocktail through brain endothelium region to maintain fluid flow and prevent cytokines from seeping into brain vasculature; maintaining segregation of the first organ region from the second organ region using the control layer; rinse the lung vasculature with fresh media; connecting the lung tissue region to the brain tissue region (e.g., with fluidic control layer 102) and flow fresh media therethrough for a period (e.g., 1 hour) of time to mimic systemic inflammation; obtain stained isolated peripheral blood mononuclear cells (PBMCs), such as with a fluorescent dye; introduce stained PBMCs into whole blood (e.g., 20% (v/v) using salt solution to reduce the hematocrit and improve visibility; and monitor the stained PBMCs in the whole blood within the vasculature of the chip over a time period and observe the PBMC adhesion and migration relative to the lung tissue and brain tissue over a period of time. After completing these steps, cytokine release and leukocyte-driven endothelial dysfunction can be measured, and anti-inflammatory agents can be delivered in circulating whole blood to assess their ability to resolve inflammation.

Vascularized tissue(s) within the multiorgan chip are exposed to an inflammatory stimulus, such as cytokines, pathogens, or chemical agents, or a defined period. The stimulus may be applied to a single tissue to serve as the primary site of inflammation, or across multiple tissues to simulate a cytokine storm. Next, the chip is flushed with fresh media and reconfigured so that all tissues are connected through a shared vascular network. Media is then circulated to simulate systemic inflammation. Fluorescently labeled PBMCs in whole blood are introduced into the vascular circuit, and effluents are collected over time. Cytokine concentrations in the effluent are measured using multiplex immunoassays or ELISA to determine the immune activation profile. Concurrently, leukocyte adhesion, transmigration, and endothelial disruption are monitored using live imaging, TEER, or permeability assays. This platform enables real-time analysis of both soluble inflammatory signals and functional endothelial responses driven by immune cell interactions.

### EMBODIMENTS

In some embodiments, a microfluidic chip can include: a plurality of separate microfluidic regions formed into a chip substrate, each microfluidic region configured to simulate an organ and comprising at least one inlet and at least one outlet with a microfluidic channel therebetween; and connecting microfluidic pathways between the separate microfluidic regions, wherein the connecting microfluidic pathways are configured to be vascularized. In some aspects, the connecting microfluidic pathways comprise a surface treatment configured to receive endothelial cells. In some aspects, the surface treatment comprises an extracellular matrix protein coating. In some aspects, endothelial cells are disposed within the connecting microfluidic pathways. In some embodiments, the endothelial cells comprise organ-specific endothelial cells that match an organ simulated by at least one of the separate microfluidic regions.

In some embodiments, the device can include: a first cell type of a first organ in a first microfluidic region configured for simulating the first organ; and a second cell type of a second organ in a second microfluidic region configured for simulating the second organ, wherein the first organ is different from the second organ. In some aspects, the connecting microfluidic pathways comprise synthetic microvascular networks (SMNs) having non-linear channels with physiologically relevant geometries. In some aspects, the physiologically relevant geometries comprise bifurcations, varying cross-sectional areas, and convolutions. In some aspects, the connecting microfluidic pathways comprise idealized microvascular networks (IMNs) having linear channels with uniform dimensions.

In some embodiments, at least one of the separate microfluidic regions comprises a porous wall between at least two microfluidic channels to allow molecular exchange while maintaining separation between different cell types.

In some embodiments, at least one electrode set is operably coupled with at least one of the separate microfluidic regions, wherein the electrode set is configured to measure trans-epithelial electrical resistance of cells. In some aspects, an oxygen sensor operably coupled with at least one of the separate microfluidic regions. In some aspects, a camera operably coupled with at least one of the separate microfluidic regions.

In some embodiments, the plurality of separate microfluidic regions comprise at least one of: a brain microfluidic region configured to simulate a blood-brain barrier, a lung microfluidic region configured to simulate an alveolar-capillary barrier, a liver microfluidic region configured to simulate hepatic metabolism, a kidney microfluidic region configured to simulate a glomerular filtration barrier, a heart microfluidic region configured to simulate myocardial tissue perfusion, a gut microfluidic region configured to simulate intestinal absorption and barrier function, a skin microfluidic region configured to simulate dermal barrier permeability, a pancreas microfluidic region configured to simulate insulin secretion, a spleen microfluidic region configured to simulate immune cell filtration, a lymph node microfluidic region configured to simulate lymphatic immune response, a bone marrow microfluidic region configured to simulate hematopoietic stem cell niches, a thymus microfluidic region configured to simulate T-cell maturation, a bladder microfluidic region configured to simulate urothelial barrier properties, a prostate microfluidic region configured to simulate androgen-responsive tissue activity, a testis microfluidic region configured to simulate the blood-testis barrier, an ovary microfluidic region configured to simulate follicular development, a placenta microfluidic region configured to simulate maternal-fetal exchange, a retina microfluidic region configured to simulate the blood-retinal barrier, a spinal cord microfluidic region configured to simulate cerebrospinal fluid dynamics, a muscle microfluidic region configured to simulate neuromuscular junctions, a breast microfluidic region configured to simulate mammary glandular secretion, a cornea microfluidic region configured to simulate ocular surface permeability, a nasal microfluidic region configured to simulate olfactory epithelium transport, a esophagus microfluidic region configured to simulate epithelial lining function, a stomach microfluidic region configured to simulate gastric secretion, a small intestine microfluidic region configured to simulate nutrient absorption, a colon microfluidic region configured to simulate microbiome interactions, a thyroid microfluidic region configured to simulate hormone secretion, a adrenal gland microfluidic region configured to simulate corticosteroid production, or a bile duct microfluidic region configured to simulate bile transport. In some aspects, each separate microfluidic region includes cells of the organ or tissue.

In some embodiments, the chip substrate includes at least one set of electrode recesses formed into the chip substrate adjacent to at least one separate microfluidic region.

In some embodiments, a microfluidic system can include: the microfluidic chip of one of the embodiments; and at least one pump operably coupled with the plurality of separate microfluidic regions to cause directional fluid flow through the separate microfluidic regions and the connecting microfluidic pathways. In some aspects, a fluidic control layer is coupled with the chip substrate so as to have at least two ports fluidly coupled with each separate microfluidic region. In some aspects, at least one pump is configured to provide either unidirectional flow or recirculating flow through the microfluidic chip. In some aspects, the microfluidic chip can include a microfluidic network with at least one bubble trap configured to prevent air bubbles from entering the microfluidic chip.

In some embodiments, a microfluidic chip can include: a substrate; a plurality of separate microfluidic regions formed on the substrate, each microfluidic region including at least one inlet and at least one outlet with a porous multi-channel assay region therebetween, each porous multi-channel assay region including at least two microfluidic channels fluidly coupled through a porous wall therebetween, wherein the microfluidic channels are shaped as synthetic microvascular networks or idealized microvascular networks, the plurality of separate microfluidic regions including a first microfluidic region and a second microfluidic region; a first connecting microfluidic pathway connecting an outlet of the first microfluidic region with an inlet of the second microfluidic region; at least one electrode set operably coupled with at least one of the microfluidic regions, wherein the electrode set is configured to be coupled with an electrode controller to measure trans-epithelial electrical resistance of cells; and a transparent cover over the substrate to define a cover for each separate microfluidic region.

In some embodiments, the microfluidic chip can include: a third microfluidic region including at least one inlet and at least one outlet with a porous multi-channel assay region therebetween, each porous multi-channel assay region including at least two microfluidic channels fluidly coupled through a porous wall therebetween, wherein the microfluidic channels are shaped as synthetic microvascular networks or idealized microvascular networks; and a second connecting microfluidic pathway connecting an outlet of the second microfluidic region to an inlet of the third microfluidic region.

In some embodiments, the microfluidic chip can include a fourth microfluidic region including at least one inlet and at least one outlet with a porous multi-channel assay region therebetween, each porous multi-channel assay region including at least two microfluidic channels fluidly coupled through a porous wall therebetween, wherein the microfluidic channels are shaped as synthetic microvascular networks or idealized microvascular networks; and a third connecting microfluidic pathway connecting an outlet of the third microfluidic region to an inlet of the fourth microfluidic region.

In some embodiments, each of the microfluidic regions includes at least one sample access port. In some aspects, at least one of the microfluidic regions includes at least one sample access port at an inlet and at least one sample access port at an outlet.

In some embodiments, at least one pump is operably coupled with the plurality of separate microfluidic regions so as to cause directional fluid flow through the first microfluidic region, through the first connecting microfluidic pathway, and through the second microfluidic region, wherein the at least one pump comprises a syringe pump or a peristaltic pump.

In some embodiments, the microfluidic chip can include two pairs of electrodes for at least two of the microfluidic regions, having a first electrode pair adjacent with and operably coupled with an inlet region, and a second electrode pair adjacent with and operably coupled with an outlet region.

In some embodiments, the microfluidic chip can include: a brain microfluidic region having at least one brain cell culture in at least one microfluidic channel; a lung microfluidic region having at least one lung cell culture in at least one microfluidic channel; and a gut microfluidic region having at least one gut cell culture in at least one microfluidic channel. In some aspects, the microfluidic chip can include: a brain microfluidic region having an astrocytes cell culture, a pericytes cell culture, and a blood vessel culture in at least two microfluidic channels; a lung microfluidic region having a bronchial epithelium cell culture and a blood vessel cell culture in at least two microfluidic channels; and a gut microfluidic region having an intestinal epithelium cell culture and a blood vessel cell culture in at least two microfluidic channels.

In some embodiments, the microfluidic chip can include one or more of: a brain microfluidic region having an apical microfluidic channel for astrocytes cell culture and pericytes cell culture, and a brain vascular microfluidic channel for blood vessel culture separated by a porous wall with pores having a width from 1 micron to 5 microns, 2 microns to 4 microns, or about 3 microns; a lung microfluidic region having a central tissue chamber with a bronchial epithelium cell culture that is surrounded by one or more lung vascular microfluidic channels having a blood vessel cell culture, with pores having a width from 3 microns to 15 microns, 6 microns to 12 microns, or 8 microns to 10 microns; and a gut microfluidic region having a gut microfluidic channel for intestinal epithelium cell culture and a gut vascular microfluidic channel for blood vessel cell culture, with pores having a width from 1 micron to 10 microns, 1.25 to 7 microns, or about 1.5 microns to about 5 microns. In some aspects, the microfluidic chip can include one or more of: the apical channel has a width from about 200 microns to about 800 microns, about 400 microns to about 600 microns, or about 500 microns; the brain vascular microfluidic channel has a width from about 50 microns to about 400 microns, 100 microns to about 300 microns or about 200 microns; the lung vascular microfluidic channel has a width from about 15 microns to about 200 microns, about 25 microns to about 175 microns, or about 50 microns to 125 microns; the central tissue chamber has a width between 200 microns to 2000 microns, about 250 microns to about 1000 microns, or greater than 500 microns; the gut microfluidic channel has a width from about 200 microns to about 800 microns, about 400 microns to about 600 microns, or about 500 microns; or the gut vascular microfluidic channel has a width from about 50 microns to about 400 microns, 100 microns to about 300 microns or about 200 microns.

In some embodiments, the microfluidic chip can include one or more of: a brain microfluidic region having an apical microfluidic channel for astrocytes cell culture and pericytes cell culture, and brain vascular microfluidic channel for blood vessel culture separated by a porous wall with pores having a width of about 3 microns, or from 2 microns to 4 microns, or 1 micron to 5 microns; a lung microfluidic region having a central tissue chamber with a bronchial epithelium cell culture that is surrounded by one or more lung vascular microfluidic channels having a blood vessel cell culture, with pores having a width of about 8 microns, or from 7 microns to 9 microns, or from 6 microns to 10 microns; or a gut microfluidic region having a gut microfluidic channel for intestinal epithelium cell culture and a gut vascular microfluidic channel for blood vessel cell culture, with pores having a width of about 3 microns, or from 2 microns to 4 microns, or 1 micron to 5 microns.

In some embodiments, the microfluidic chip can include: the apical channel having a width of about 500 microns, or from about 450 microns to about 550 microns, or about 400 microns to about 600 microns; the brain vascular microfluidic channel(s) having a width of about 200 microns, or about 150 microns to about 250 microns, or about 100 microns to about 300 microns; the lung vascular microfluidic channel(s) having a width of about 100 microns, or about 50 microns to about 150 microns, or about 25 microns to about 175 microns; the central tissue chamber (e.g., lung) having a width of greater than 500 microns; the gut microfluidic channel having a width of about 500 microns, or from about 450 microns to about 550 microns, or about 400 microns to about 600 microns; and the gut vascular microfluidic channel(s) having a width of about 200 microns, or about 150 microns to about 250 microns, or about 100 microns to about 300 microns.

In some embodiments, the microfluidic chip can include a measuring device configured to measure current and voltage at the at least one electrode pair. In some aspects, the substrate includes an electrode recess shaped to accommodate each electrode of the at least one electrode pair. In some aspects, there are four electrodes, wherein two electrodes input a current signal and two electrodes detect a voltage signal across a cell layer. In some aspects, the microfluidic chip can include: the substrate having electrode recesses and electrode lead conduits formed therein, with electrodes and electrode leads therein; a microfluidic layer having the microfluidic regions; and a fluidics control layer having inlets and outlets that are operably coupled with inlets and outlets of the microfluidic regions of the microfluidic layer.

In some embodiments, a microfluidic chip system can include: the microfluidic chip of one of the embodiments; at least one camera; and a computer operably coupled with the camera so as to record video or still images. In some aspects, one camera views a plurality of the microfluidic regions, or each microfluidic region includes a unique camera.

In some embodiments, a microfluidic chip can include: a substrate; a plurality of separate microfluidic regions formed on the substrate, each microfluidic region including at least one inlet and at least one outlet with a porous multi-channel assay region therebetween, each porous multi-channel assay region including at least two microfluidic channels fluidly coupled through a porous wall therebetween, wherein the microfluidic channels are shaped as synthetic microvascular networks or idealized microvascular networks, the plurality of separate microfluidic regions including a first microfluidic region and a second microfluidic region; a first connecting microfluidic pathway connecting an outlet of the first microfluidic region with an inlet of the second microfluidic region; an oxygen sensor operably coupled with at least one of the microfluidic regions; and a transparent cover over the substrate to define a cover for each separate microfluidic region. In some aspects, the first microfluidic region comprises a lung microfluidic region configured for air-liquid interface culture. In some embodiments, the first microfluidic region comprises a heart microfluidic region and the second microfluidic region comprises a skeletal muscle microfluidic region, and wherein the first connecting microfluidic pathway comprises progressively narrowing bifurcations configured to simulate physiological blood flow.

In some embodiments, a microfluidic chip formed on a substrate can include: a plurality of separate microfluidic regions, each microfluidic region including at least one inlet and at least one outlet with a porous multi-channel assay region therebetween, each porous multi-channel assay region including at least two microfluidic channels fluidly coupled through at porous wall therebetween, wherein optionally at least one microfluidic channel is configured as tissue chamber, wherein the microfluidic channels are shaped as synthetic microvascular networks or idealized microvascular networks, the plurality of separate microfluidic regions including a first microfluidic region and a second microfluidic region; a first connecting microfluidic pathway connecting an outlet of the first microfluidic region with an inlet of the second microfluidic region; and a cover over the substrate to define a cover for each separate microfluidic region, wherein the cover is transparent. In some aspects, at least one electrode pair is operably coupled with at least one of the microfluidic regions, wherein the electrode pair is configured to be coupled with an electrode controller to measure trans-epithelial electrical resistance of cells.

In some embodiments, a third microfluidic region includes at least one inlet and at least one outlet with a porous multi-channel assay region therebetween, each porous multi-channel region including at least two microfluidic channels fluidly coupled through at porous wall therebetween, wherein optionally at least one microfluidic channel is configured as tissue chamber, wherein the microfluidic channels are shaped as synthetic microvascular networks or idealized microvascular networks; and a second connecting microfluidic pathway connecting an outlet of the second microfluidic region to an inlet of the third microfluidic region. In some aspects, a fourth microfluidic region includes at least one inlet and at least one outlet with a porous multi-channel assay region therebetween, each porous multi-channel region including at least two microfluidic channels fluidly coupled through at porous wall therebetween, wherein optionally at least one microfluidic channel is configured as tissue chamber, wherein the microfluidic channels are shaped as synthetic microvascular networks or idealized microvascular networks; and a third connecting microfluidic pathway connecting an outlet of the third microfluidic region to an inlet of the fourth microfluidic region.

In some embodiments, a method of assaying a multi-organ response is provided. The method can include: providing a microfluidic chip comprising a plurality of separate microfluidic regions configured to simulate different organs and connecting microfluidic pathways between the separate microfluidic regions, wherein the connecting microfluidic pathways are vascularized with endothelial cells; introducing a test agent into a first microfluidic region of the plurality of separate microfluidic regions; circulating fluid through the connecting microfluidic pathways to transport the test agent or cellular products from the first microfluidic region to a second microfluidic region; and monitoring a response in the second microfluidic region.

In some embodiments, monitoring the response comprises measuring at least one of: trans-epithelial electrical resistance; oxygen consumption; cellular morphology; barrier integrity; cytokine release; cell viability; or immune cell adhesion and migration.

In some embodiments, the test agent comprises at least one of: a visual particle; a cell; an inflammatory agent; an anti-inflammatory agent; a drug; a toxin; an antigen; or an infectious agent.

In some embodiments, the method can include: determining a biological response of the cells in the first microfluidic region to the test agent; and determining a biological response of the cells in the second microfluidic region to the test agent or the one or more analytes.

In some embodiments, the method can include: obtaining data from the microfluidic chip, wherein the data comprises an image, a video, a voltage, an oxygen sensor reading, a temperature, or a flow rate; and recording the data to a non-transitory memory device.

In some embodiments, the method can include controlling a flow rate and a temperature of fluid in the microfluidic chip with a computing device.

In some embodiments, the method can include monitoring tissue barrier integrity and oxygen consumption during exposure to the test agent.

In some embodiments, the method can include: obtaining a fluid sample from the microfluidic chip; and assaying the fluid sample for presence of an analyte. In some aspects, the analyte includes the analyte is selected from the group consisting of a protein, a peptide, a nucleic acid, a hormone, a cytokine, a chemokine, a lipid, a metabolite, a carbohydrate, an electrolyte, a drug, a toxin, a pathogen-associated molecular pattern (PAMP), a cell-free DNA fragment, a biomarker of inflammation, a biomarker of infection, a biomarker of organ dysfunction, a therapeutic agent, and a combination thereof. In some aspects, the analyte is measured by n assay selected from the group consisting of an immunoassay, an enzyme-linked immunosorbent assay (ELISA), a radioimmunoassay, a lateral flow assay, a chemiluminescent assay, a fluorescence-based assay, a colorimetric assay, a nucleic acid amplification assay, a polymerase chain reaction (PCR) assay, a quantitative PCR (qPCR) assay, a digital PCR assay, a microarray assay, a next-generation sequencing (NGS) assay, a mass spectrometry-based assay, a surface plasmon resonance (SPR) assay, an electrochemical assay, a biosensor-based assay, a label-free detection assay, a magnetic bead-based assay, a flow cytometry assay, and a combination thereof.

### EXAMPLES

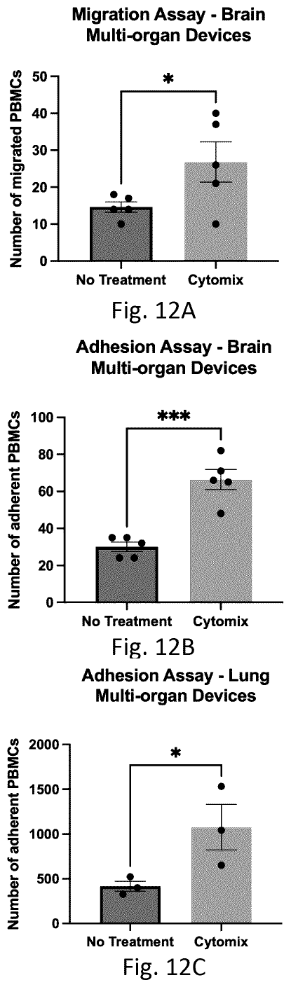

The device can be used for various assays to study the differ organs in response to stimuli, and response of subsequent organs to a prior organ response. As such, migration and adhesion assays can be performed on the multi-organ devices after inducing inflammation using Cytomix, a cytokine cocktail (mix of cytokines) used to induce inflammation in cells. The different organs in the multiorgan chip can be studied to study number of migrated PMBCs when there is no treatment versus the cytokine cocktail. The exemplified organs in this study include brain, and lung. For the brain, migration and adhesion assays are conducted. For the lung, adhesion assays are conducted. The data is shown in the graphs of FIGS. 12A-12C.

FIGS. 12A-12B include graphs that show data outputs for migration and adhesion assays in brain tissue. FIG. 12C includes a graph with data for the adhesion assay output in lung tissue of the multi-organ devices. The data shows a significant increase in PBMCs that migrated to the brain and have adhesion to the bran and lung compared to no treatment.

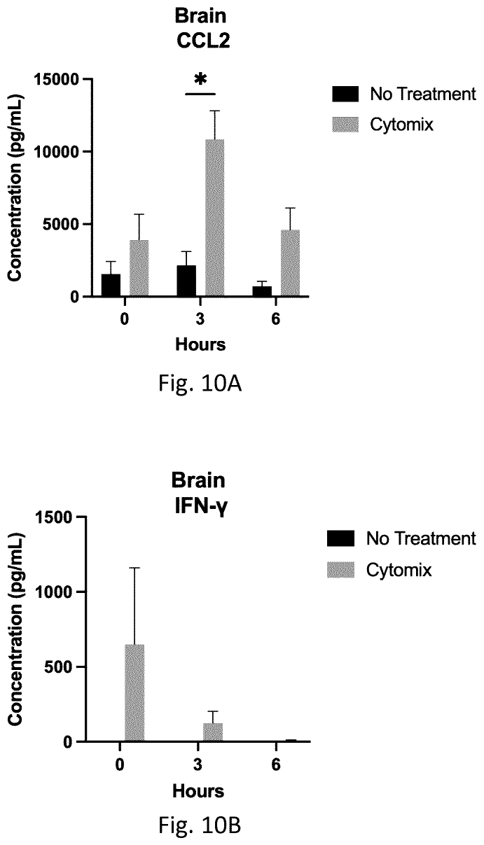

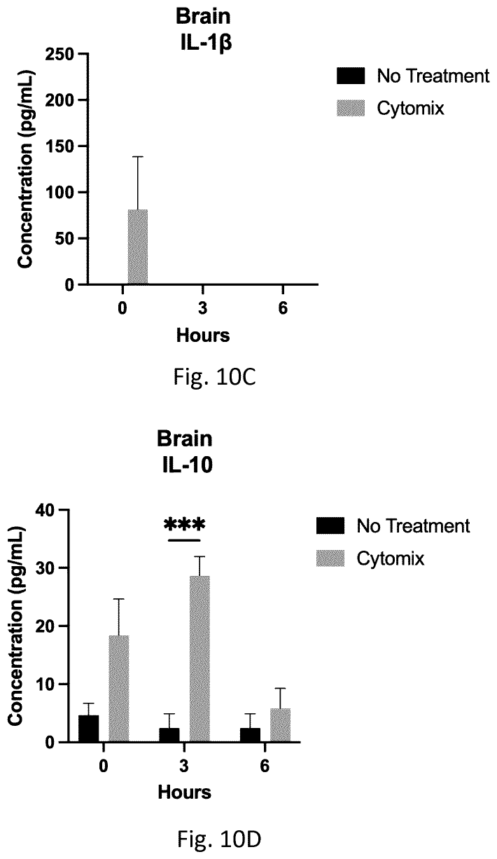

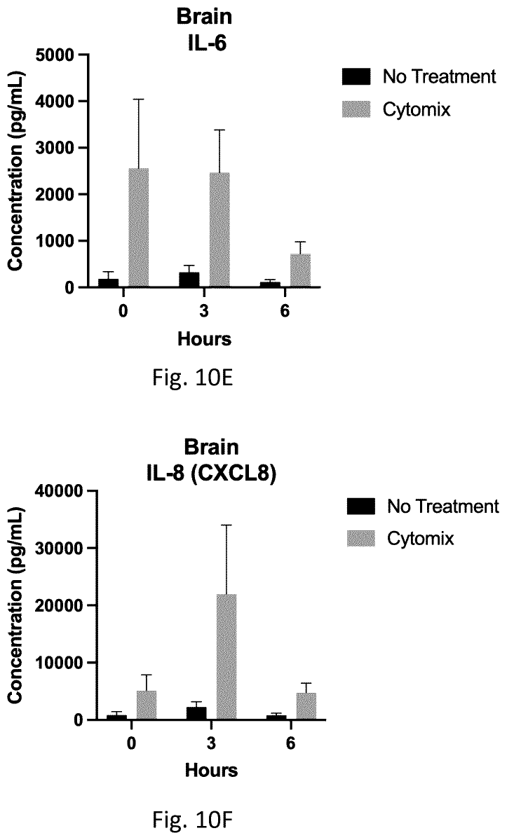

FIGS. 10A-10F include graphs that show levels of cytokines and chemokines measured in effluents collected from brain tissues of the multi-organ devices after inducing inflammation using Cytomix.

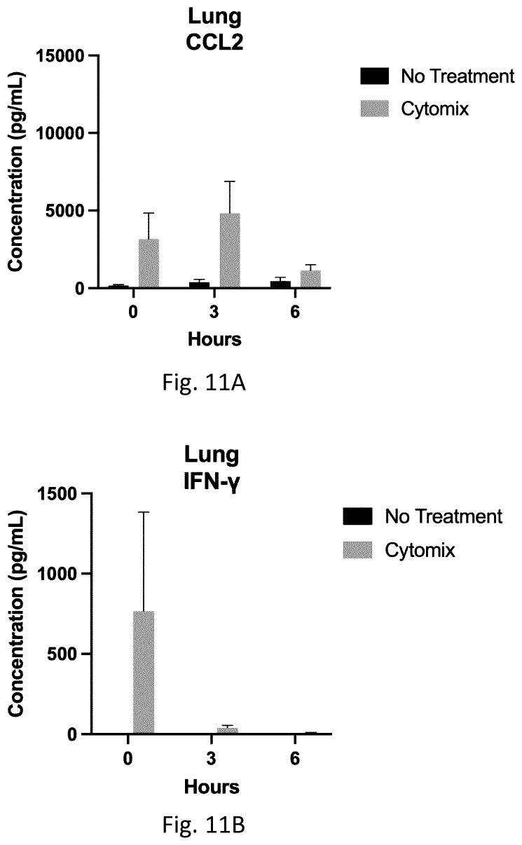

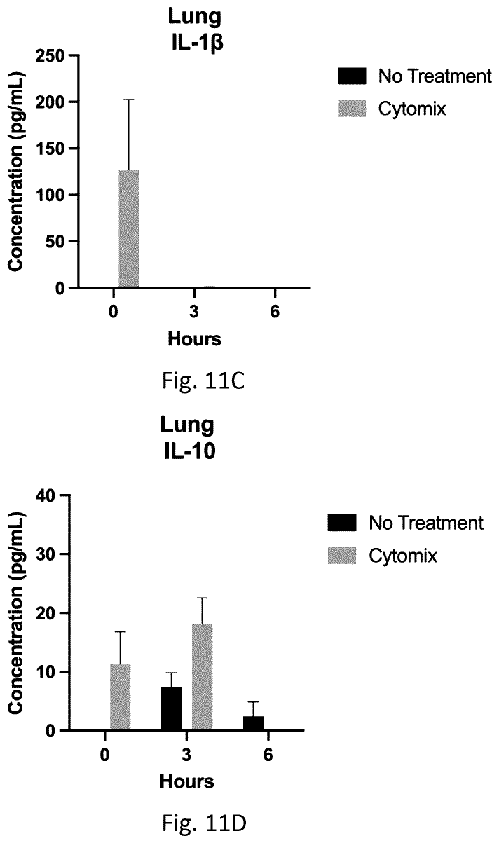

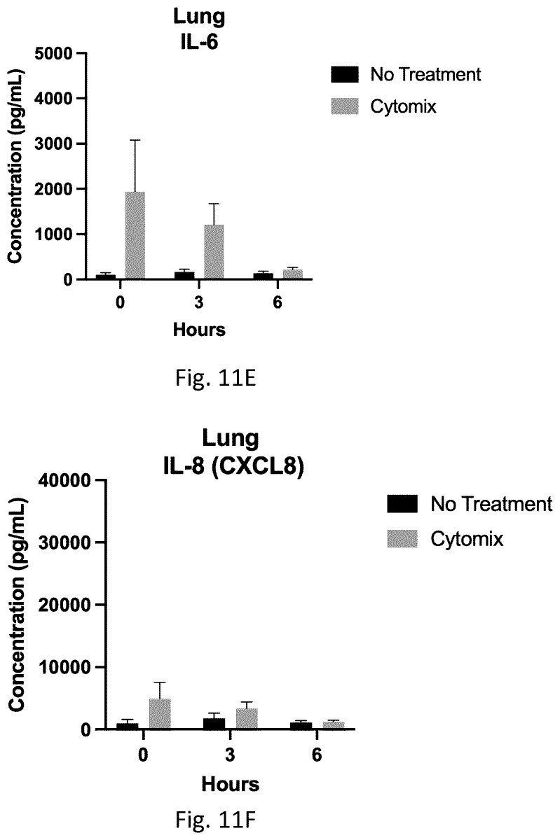

FIGS. 11A-11F include graphs that show levels of cytokines and chemokines measured in effluents collected from lung tissues of the multi-organ devices after inducing inflammation using Cytomix.

The cytokine data shows that the cytokine mixture increases levels of cytokines in the brain and lung for the different measured cytokines. This shows that the multiorgan device can be used to measure responses of multiple organs to a stimulus, such as induced inflammation.

### Methods and Computations

One skilled in the art can appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more protocols or algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

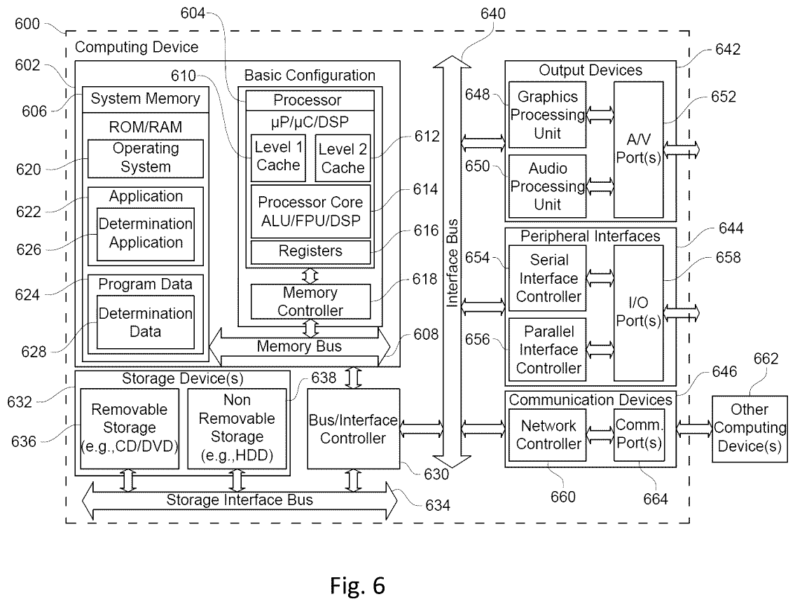

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method described herein. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

### Definitions

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

A “synthetic microvascular network” (SMN) is an engineered network of interconnected, nonlinear flow channels that mimic the geometrical features and fluid flow properties found in physiological microvascular networks. These channels form an intersecting network, which can be arranged to replicate the sequence of arterioles, capillaries, and venules. Each flow channel in an SMN exhibits one or more characteristics of physiological microvascular vessels, such as variable cross-sectional shapes, varying cross-sectional areas, convolutions, turns, and anastomoses. A network of linear flow channels joining at fixed angles, for example, does not qualify as an SMN. Straight channels or those with non-physiological geometries may be used to connect the synthetic microvascular network to other components of a microfluidic chip, but these channels are not considered part of the microvascular network itself.

The term “idealized” when referring to a microfluidic network, junction, or bifurcation describes a synthetic network, junction, or bifurcation consisting of straight microfluidic channels joined at acute, right, or obtuse angles.

A “microfluidic channel” may have a rectangular, circular, semi-circular, irregular, or a combination of cross-sectional shapes. The dimensions of a channel are described by length, depth, and width, where the depth is measured perpendicular to the plane of the microfluidic chip containing the channel, and length and width are measured within the plane of the chip. Channels with circular or semi-circular cross-sections may be described as having variable depth and width, or alternatively, may be described by their diameter. When referring to a channel with a circular or semi-circular cross-section, the maximum depth and width are both equal to the maximum diameter of the channel. For channels with a rectangular cross-section, the maximum width and depth refer to the constant width and depth of a channel with uniform dimensions, or to the highest values for width and depth in channels with varying dimensions.

A “microfluidic chip” is constructed using techniques common in the semiconductor industry, such as photolithography, wet chemical etching, thin film deposition, laser patterning, and soft lithography with polymeric substrates. This differs from microfluidic systems made using gels composed of proteins, chitosan, proteoglycans, or other extracellular matrix components. In general, a microfluidic chip contains multiple microchannels that are connected to various reservoirs holding fluid materials. These fluids are driven or displaced through the microchannels using electrokinetic forces, pumps, or other mechanisms.

All references recited herein are incorporated herein by specific reference in their entirety, including: U.S. Pat. Nos. 7,189,578; 7,604,394; 8,147,775; 9,283,597; 9,878,090; 8,828,715; 7,725,267; 8,175,814; 8,589,083; 10,012,640; 9,932,550; 10,570,360; 8,380,443; 8,940,494; 8,417,465; 8,355,876; 9,453,252; 9,933,413; 10,641,761; 9,291,614; 9,784,727, 10,775,364; US 2007/0231783; US 2010/0112550; US 2013/0149735; US 2010/0227312; US 2015/0377861; US 2015/0299631; and US 2014/0255961.

1. A microfluidic chip comprising:
a plurality of separate microfluidic regions formed into a chip substrate, each microfluidic region configured to simulate an organ and comprising at least one inlet and at least one outlet with a microfluidic channel therebetween; and
connecting microfluidic pathways between the separate microfluidic regions, wherein the connecting microfluidic pathways are configured to be vascularized.

2. The microfluidic chip of claim 1, wherein the connecting microfluidic pathways comprise a surface treatment configured to receive endothelial cells.

3. The microfluidic chip of claim 2, wherein the surface treatment comprises an extracellular matrix protein coating.

4. The microfluidic chip of claim 2, further comprising endothelial cells disposed within the connecting microfluidic pathways.

5. The microfluidic chip of claim 4, wherein the endothelial cells comprise organ-specific endothelial cells that match an organ simulated by at least one of the separate microfluidic regions.

6. The microfluidic chip of claim 4, further comprising:
a first cell type of a first organ in a first microfluidic region configured for simulating the first organ; and
a second cell type of a second organ in a second microfluidic region configured for simulating the second organ,
wherein the first organ is different from the second organ.

7. The microfluidic chip of claim 1, wherein the connecting microfluidic pathways comprise synthetic microvascular networks (SMNs) having non-linear channels with physiologically relevant geometries.

8. The microfluidic chip of claim 7, wherein the physiologically relevant geometries comprise bifurcations, varying cross-sectional areas, and convolutions.

9. The microfluidic chip of claim 1, wherein the connecting microfluidic pathways comprise idealized microvascular networks (IMNs) having linear channels with uniform dimensions.

10. The microfluidic chip of claim 1, wherein at least one of the separate microfluidic regions comprises a porous wall between at least two microfluidic channels to allow molecular exchange while maintaining separation between different cell types.

11. The microfluidic chip of claim 1, further comprising at least one electrode set operably coupled with at least one of the separate microfluidic regions, wherein the electrode set is configured to measure trans-epithelial electrical resistance of cells.

12. The microfluidic chip of claim 1, further comprising an oxygen sensor operably coupled with at least one of the separate microfluidic regions.

13. The microfluidic chip of claim 1, further comprising a camera operably coupled with at least one of the separate microfluidic regions.

14. The microfluidic chip of claim 1, wherein the plurality of separate microfluidic regions comprise at least one of: a brain microfluidic region configured to simulate a blood-brain barrier, a lung microfluidic region configured to simulate an alveolar-capillary barrier, a liver microfluidic region configured to simulate hepatic metabolism, a kidney microfluidic region configured to simulate a glomerular filtration barrier, a heart microfluidic region configured to simulate myocardial tissue perfusion, a gut microfluidic region configured to simulate intestinal absorption and barrier function, a skin microfluidic region configured to simulate dermal barrier permeability, a pancreas microfluidic region configured to simulate insulin secretion, a spleen microfluidic region configured to simulate immune cell filtration, a lymph node microfluidic region configured to simulate lymphatic immune response, a bone marrow microfluidic region configured to simulate hematopoietic stem cell niches, a thymus microfluidic region configured to simulate T-cell maturation, a bladder microfluidic region configured to simulate urothelial barrier properties, a prostate microfluidic region configured to simulate androgen-responsive tissue activity, a testis microfluidic region configured to simulate the blood-testis barrier, an ovary microfluidic region configured to simulate follicular development, a placenta microfluidic region configured to simulate maternal-fetal exchange, a retina microfluidic region configured to simulate the blood-retinal barrier, a spinal cord microfluidic region configured to simulate cerebrospinal fluid dynamics, a muscle microfluidic region configured to simulate neuromuscular junctions, a breast microfluidic region configured to simulate mammary glandular secretion, a cornea microfluidic region configured to simulate ocular surface permeability, a nasal microfluidic region configured to simulate olfactory epithelium transport, a esophagus microfluidic region configured to simulate epithelial lining function, a stomach microfluidic region configured to simulate gastric secretion, a small intestine microfluidic region configured to simulate nutrient absorption, a colon microfluidic region configured to simulate microbiome interactions, a thyroid microfluidic region configured to simulate hormone secretion,
a adrenal gland microfluidic region configured to simulate corticosteroid production, or a bile duct microfluidic region configured to simulate bile transport.

15. The microfluidic chip of claim 14, wherein each separate microfluidic region includes cells of the organ.

16. The microfluidic chip of claim 1, further comprising butterfly ports between the separate microfluidic regions, each butterfly port including at least two port lobes, a fluidic connector.

17. The microfluidic chip of claim 1, comprising at least three separate microfluidic regions that are interconnected by the connecting microfluidic pathways that are configured to be vascularized.

18. The microfluidic chip of claim 1, comprising at least one set of electrode recesses formed into the chip substrate adjacent to at least one separate microfluidic region.

19. A microfluidic system comprising:
the microfluidic chip of claim 1; and
at least one pump operably coupled with the plurality of separate microfluidic regions to cause directional fluid flow through the separate microfluidic regions and the connecting microfluidic pathways.

20. The microfluidic system of claim 19, further comprising a fluidic control layer that is coupled with the chip substrate so as to have at least two ports fluidly coupled with each separate microfluidic region.

21. The microfluidic system of claim 19, wherein the at least one pump is configured to provide either unidirectional flow or recirculating flow through the microfluidic chip.

22. The microfluidic system of claim 19, further comprising at least one bubble trap configured to prevent air bubbles from entering the microfluidic chip.

23. A method of assaying a multi-organ response, comprising:
providing a microfluidic chip comprising a plurality of separate microfluidic regions configured to simulate different organs and connecting microfluidic pathways between the separate microfluidic regions, wherein the connecting microfluidic pathways are vascularized with endothelial cells;
introducing a test agent into a first microfluidic region of the plurality of separate microfluidic regions;
circulating fluid through the connecting microfluidic pathways to transport the test agent or cellular products from the first microfluidic region to a second microfluidic region; and
monitoring a response in the second microfluidic region.

24. The method of claim 23, wherein monitoring the response comprises measuring at least one of:
trans-epithelial electrical resistance;
oxygen consumption;
cellular morphology;
barrier integrity;
cytokine release;
cell viability; or
immune cell adhesion and migration.

25. The method of claim 23, wherein the test agent comprises at least one of:
a visual particle;
a cell;
an inflammatory agent;
an anti-inflammatory agent;
a drug;
a toxin;
an antigen;
particle;
nanoparticle;
ribonuclear protein (RNP) complex;
virus;
viral particle;
bacteria;
fungus;
or
an infectious agent.

26. The method of claim 23, further comprising:
determining a biological response of the cells in the first microfluidic region to the test agent; and
determining a biological response of the cells in the second microfluidic region to the test agent or the one or more analytes.

27. The method of claim 23, further comprising:
obtaining data from the microfluidic chip, wherein the data comprises an image, a video, a voltage, an oxygen sensor reading, a temperature, or a flow rate; and
recording the data to a non-transitory memory device.

28. The method of claim 23, further comprising controlling a flow rate and a temperature of fluid in the microfluidic chip with a computing device.

29. The method of claim 23, further comprising monitoring tissue barrier integrity and oxygen consumption during exposure to the test agent.

30. The method of claim 23, further comprising:
obtaining a fluid sample from the microfluidic chip; and
assaying the fluid sample for presence of an analyte.

31. The method of claim 30, wherein the analyte includes the analyte is selected from the group consisting of a protein, a peptide, a nucleic acid, a hormone, a cytokine, a chemokine, a lipid, a metabolite, a carbohydrate, an electrolyte, a drug, a toxin, a pathogen-associated molecular pattern (PAMP), a cell-free DNA fragment, a biomarker of inflammation, a biomarker of infection, a biomarker of organ dysfunction, a therapeutic agent, and a combination thereof.

32. The method of claim 30, wherein the analyte is measured by n assay selected from the group consisting of an immunoassay, an enzyme-linked immunosorbent assay (ELISA), a radioimmunoassay, a lateral flow assay, a chemiluminescent assay, a fluorescence-based assay, a colorimetric assay, a nucleic acid amplification assay, a polymerase chain reaction (PCR) assay, a quantitative PCR (qPCR) assay, a digital PCR assay, a microarray assay, a next-generation sequencing (NGS) assay, a mass spectrometry-based assay, a surface plasmon resonance (SPR) assay, an electrochemical assay, a biosensor-based assay, a label-free detection assay, a magnetic bead-based assay, a flow cytometry assay, and a combination thereof.
